(19) United States
(12) Patent Application Publication
(10) Pub. No.: US 2015/0327839 A1
(43) Pub. Date: Nov. 19, 2015

(54) ULTRASONIC PROBE AND ULTRASONIC DIAGNOSTIC APPARATUS

(71) Applicant: SAMSUNG ELECTRONICS CO., LTD., Suwon-si (KR)

(72) Inventors: Baehyung KIM, Yongin-si (KR); Youngil KIM, Suwon-si (KR); Jong Keun SONG, Yongin-si (KR); Seungheun LEE, Seongnam-si (KR); Kyungil CHO, Seoul (KR)

(73) Assignee: SAMSUNG ELECTRONICS CO., LTD., Suwon-si (KR)

(21) Appl. No.: 14/711,968

(22) Filed: May 14, 2015

(30) Foreign Application Priority Data
May 14, 2014 (KR) 10-2014-0057714

(51) Int. Cl. A61B 8/00; A61B 8/08; H02J 7/02; A61B 8/14

(52) U.S. Cl. CPC A61B 8/4433; A61B 8/14; A61B 8/4472; A61B 8/4488; A61B 8/56; A61B 8/5207; H02J 7/025

(57) ABSTRACT

Disclosed herein is an ultrasonic diagnostic apparatus including: an ultrasonic probe including an ultrasonic transducer array; and an ultrasonic diagnostic apparatus main body comprising a transceiver configured to transmit and receive ultrasonic waves via the ultrasonic transducer array, an image processor configured to generate an ultrasonic image of an object based on an ultrasonic echo signal acquired via the transceiver, a communication unit configured to wirelessly communicate with a docking station, and a charge unit configured to charge power which is received wirelessly from the docking station via the communication unit, in a charge battery. Therefore, it is possible to efficiently supply power to the ultrasonic diagnostic apparatus main body regardless of time and place, and to improve mobility and portability of the ultrasonic diagnostic apparatus main body.

### CROSS-REFERENCE TO RELATED APPLICATION

This application claims priority from Korean Patent Application No. 10-2014-0057714, filed on May 14, 2014 in the Korean Intellectual Property Office, the disclosure of which is incorporated herein by reference in its entirety.

### BACKGROUND

1. Field

Exemplary embodiments relate to an ultrasonic probe, an ultrasonic diagnostic apparatus, and an ultrasonic diagnostic system.

2. Description of the Related Art

An ultrasonic diagnostic apparatus irradiates ultrasonic signals to a target region of an object from the surface of the object, and receives ultrasonic signals (ultrasonic echo signals) reflected from the target region so as to non-invasively acquire section images about soft tissue of the object or images about blood vessels of the object based on the echo ultrasonic signals. The ultrasonic diagnostic apparatus has advantages that it is a compact, low-priced apparatus and it can display images in real time, compared to other medical imaging apparatuses, such as an X-ray diagnostic apparatus, an X-ray Computerized Tomography (CT) scanner, a Magnetic Resonance Image (MRI) apparatus, and a nuclear medical diagnostic apparatus. Also, the ultrasonic diagnostic apparatus has high safety since there is no risk for patients to be exposed to radiation such as X-rays. For the advantages, the ultrasonic diagnostic apparatus is widely used to diagnose the heart, abdomen, urinary organs, uterus, etc.

Typically, an ultrasonic diagnostic apparatus is fixed and used at a specific place since it is large and heavy, and when an ultrasonic diagnostic apparatus needs to be moved, a cart type ultrasonic diagnostic apparatus having castors is generally used. Recently, a portable ultrasonic diagnostic apparatus with a compact size and light weight has been developed and used.

In order to move the cart type ultrasonic diagnostic apparatus which is large and heavy, it is imperative to unplug a power plug connected to a wired power cable of the apparatus from an electrical outlet, to move the ultrasonic diagnostic apparatus, and then to plug the power plug in an electrical outlet at a destination place to supply power to the ultrasonic diagnostic apparatus. However, since it takes a long time to move the ultrasonic diagnostic apparatus, to plug the power plug in the electrical outlet at the destination place (for example, an operating room), and then to reboot the ultrasonic diagnostic apparatus, there is a risk that power may not be quickly and stably supplied to the ultrasonic diagnostic apparatus in an emergency situation, such as a surgery or a treatment of an emergency patient. Although there is a method of installing an emergency power source (battery) in the ultrasonic diagnostic apparatus so that the ultrasonic diagnostic apparatus can be used without rebooting for a predetermined time, the time for which the ultrasonic diagnostic apparatus can be used without rebooting is limited, and it is inconvenient that it is still imperative to plug a power plug connected to a wired power cable of the apparatus in an electrical outlet at a destination place after moving the apparatus, in order to stably operate the ultrasonic diagnostic apparatus.

Meanwhile, the portable ultrasonic diagnostic apparatus has an advantage that it can be conveniently moved, since it is compact and light-weight. However, a power supply technique for reducing the size and weight of the portable ultrasonic diagnostic apparatus by installing a battery of a smaller volume still needs to be developed.

### SUMMARY

Therefore, it is an aspect of one or more exemplary embodiments to provide an ultrasonic probe and an ultrasonic diagnostic apparatus, capable of efficiently supplying power to the ultrasonic probe and an ultrasonic diagnostic apparatus main body regardless of time and place, by applying a wireless power transfer technique to the ultrasonic probe and the ultrasonic diagnostic apparatus main body.

It is another aspect of one or more exemplary embodiments to provide an ultrasonic probe and an ultrasonic diagnostic apparatus, capable of improving mobility and portability of the ultrasonic probe and an ultrasonic diagnostic apparatus main body and increasing use times of the ultrasonic probe and the ultrasonic diagnostic apparatus main body, by applying a wireless power transfer technique to the ultrasonic probe and the ultrasonic diagnostic apparatus main body.

Further, it is still another aspect of one or more exemplary embodiments to provide an ultrasonic probe and an ultrasonic diagnostic apparatus, capable of installing charge batteries of smaller volumes in the ultrasonic probe and an ultrasonic diagnostic apparatus main body to reduce sizes and weights of the ultrasonic probe and the ultrasonic diagnostic apparatus main body, by applying a wireless power transfer technique to the ultrasonic probe and the ultrasonic diagnostic apparatus main body.

Additional aspects of the exemplary embodiments will be set forth in part in the description which follows and, in part, will be obvious from the description, or may be learned by practice of the exemplary embodiments.

In accordance with one aspect of one or more exemplary embodiments, an ultrasonic diagnostic apparatus includes: an ultrasonic probe including an ultrasonic transducer array; and an ultrasonic diagnostic apparatus main body comprising a transceiver configured to transmit and receive ultrasonic waves via the ultrasonic transducer array, an image processor configured to generate an ultrasonic image of an object based on an ultrasonic echo signal acquired via the transceiver, a communicator configured to wirelessly communicate with a docking station, and a charger configured to charge power which is wirelessly received from the docking station via the communicator, in a charge battery.

The ultrasonic diagnostic apparatus main body may further include a power supply controller configured to control power supplied from an external device, wherein the power supply controller may be further configured to receive power which is transmitted wirelessly from the docking station, and to transfer the received power to the charger.

The communicator may be further configured to wirelessly transmit the ultrasonic echo signal and the ultrasonic image to the docking station.

The charger may be further configured to charge the power received from the docking station, in the charge battery, by using at least one method from among a capacitive method using an electric field, a resonance method using a magnetic field, and an inductive method.

The ultrasonic diagnostic apparatus main body may further include: a battery level calculator configured to calculate a battery level of the charge battery; and a display configured to display the calculated battery level of the charge battery and the ultrasonic image.

The ultrasonic diagnostic apparatus main body may further include an input device configured to set a wireless power transfer mode for wirelessly receiving power from the docking station.

In accordance with another aspect of one or more exemplary embodiments, an ultrasonic diagnostic apparatus includes an ultrasonic probe and an ultrasonic diagnostic apparatus main body, wherein the ultrasonic probe includes an ultrasonic transducer array, a transceiver configured to transmit and receive ultrasonic waves via the ultrasonic transducer array, a first communicator configured to wirelessly communicate with the ultrasonic diagnostic apparatus main body, and a charger configured to charge power which is wirelessly received from the ultrasonic diagnostic apparatus main body via the first communicator, in a charge battery, and wherein the ultrasonic diagnostic apparatus main body comprises a second communicator configured to wirelessly communicate with the ultrasonic probe, and an image processor configured to generate an ultrasonic image of an object based on an ultrasonic echo signal acquired via wireless communication with the ultrasonic probe.

The ultrasonic probe may further includes a power supply controller configured to control power supplied from an external device, wherein the power supply controller is further configured to receive power which is transmitted wirelessly from the ultrasonic diagnostic apparatus main body, and to transfer the received power to the charger.

The first communicator may be further configured to wirelessly transmit the ultrasonic echo signal to the ultrasonic diagnostic apparatus main body.

The ultrasonic probe may further include: a battery level calculator configured to calculate a battery level of the charge battery; and a display configured to display the calculated battery level of the charge battery.

In accordance with another aspect of one or more exemplary embodiments, an ultrasonic diagnostic apparatus includes an ultrasonic probe and an ultrasonic diagnostic apparatus main body, wherein the ultrasonic probe comprises an ultrasonic transducer array, a transceiver configured to transmit and receive ultrasonic waves via the ultrasonic transducer array, a probe communicator configured to wirelessly communicate with the ultrasonic diagnostic apparatus main body, and a probe charger configured to charge power which is wirelessly received from the ultrasonic diagnostic apparatus main body via the probe communicator, in a probe charge battery, and wherein the ultrasonic diagnostic apparatus main body comprises a first main body communicator configured to wirelessly communicate with the ultrasonic probe, an image processor configured to generate an ultrasonic image of an object based on an ultrasonic echo signal acquired from the ultrasonic probe via the first main body communicator, a second main body communicator configured to wirelessly communicate with a docking station, and a main body charger configured to charge power which is wirelessly received from the docking station via the second main body communicator, in a main body charge battery.

The ultrasonic probe may further include a probe power supply controller configured to control power supplied from an external device, wherein the probe power supply controller is further configured to receive power which is transmitted wirelessly from the ultrasonic diagnostic apparatus main body, and to transfer the received power to the probe charger.

The ultrasonic diagnostic apparatus main body may further include a main body power supply controller configured to control power supplied from an external device, wherein the main body power supply controller is further configured to receive power which is transmitted wirelessly from the docking station, and to transfer the received power to the main body charger.

The probe communicator may be further configured to wirelessly transmit the ultrasonic echo signal to the ultrasonic diagnostic apparatus main body.

The second main body communicator may be further configured to wirelessly transmit the ultrasonic echo signal and the ultrasonic image to the docking station.

The ultrasonic probe may further include: a probe battery level calculator configured to calculate a battery level of the probe charge battery; and a probe display configured to display the calculated battery level of the probe charge battery.

The ultrasonic diagnostic apparatus main body may further include: a main body battery level calculator configured to calculate a battery level of the main body charge battery; and a main body display configured to display the calculated battery level of the main body charge battery.

In accordance with another aspect of one or more exemplary embodiments, an ultrasonic probe includes: an ultrasonic transducer array; a transceiver configured to transmit and receive ultrasonic waves via the ultrasonic transducer array; an image processor configured to generate an ultrasonic image of an object based on an ultrasonic echo signal acquired via the transceiver; a display configured to display the ultrasonic image of the object; and a communicator configured to wirelessly communicate with a docking station; and a charger configured to charge power which is wirelessly received from the docking station via the communicator, in a charge battery.

The ultrasonic probe may further include a power supply controller configured to control power supplied from an external device, wherein the power supply controller is further configured to receive power which is transmitted wirelessly from the docking station, and to transfer the received power to the charger.

The communicator may be further configured to wirelessly transmit the ultrasonic echo signal and the ultrasonic image to the docking station.

### BRIEF DESCRIPTION OF THE DRAWINGS

These and/or other aspects will become apparent and more readily appreciated from the following description of exemplary embodiments, taken in conjunction with the accompanying drawings of which:

FIG. 1 is a perspective view illustrating an external appearance of a cart type ultrasonic diagnostic apparatus;

FIG. 2 is a perspective view illustrating an external appearance of a portable ultrasonic diagnostic apparatus;

FIGS. 3A and 3B are views for describing an external structure of a handheld ultrasonic diagnostic apparatus;

FIG. 4A is a control block diagram of an ultrasonic diagnostic system, and FIG. 4B is a control block diagram illustrating configurations of an ultrasonic probe, an ultrasonic diagnostic apparatus main body, and a docking system shown in FIG. 4A;

FIG. 5A is a control block diagram of an ultrasonic diagnostic system, and FIG. 5B is a control block diagram illustrating configurations of ultrasonic probes, ultrasonic diagnostic apparatus main bodies, and a docking system shown in FIG. 5A;

FIG. 6A is a control block diagram of an ultrasonic diagnostic apparatus, and FIG. 6B is a control block diagram illustrating configurations of an ultrasonic probe and an ultrasonic diagnostic apparatus main body shown in FIG. 6A;

FIG. 7A is a control block diagram of an ultrasonic diagnostic system, and FIG. 7B is a control block diagram illustrating configurations of an ultrasonic probe, an ultrasonic diagnostic apparatus main body, and a docking station shown in FIG. 7A;

FIG. 8A is a control block diagram of an ultrasonic diagnostic system, and FIG. 8B is a control block diagram illustrating configurations of an ultrasonic probe and a docking station shown in FIG. 8A;

FIG. 9A is a control block diagram of an ultrasonic diagnostic system, and FIG. 9B is a control block diagram illustrating configurations of ultrasonic probes and a docking system shown in FIG. 9A; and

FIG. 10 illustrates an internal structure of an ultrasonic probe.

### DETAILED DESCRIPTION

Reference will now be made in detail to exemplary embodiments, examples of which are illustrated in the accompanying drawings.

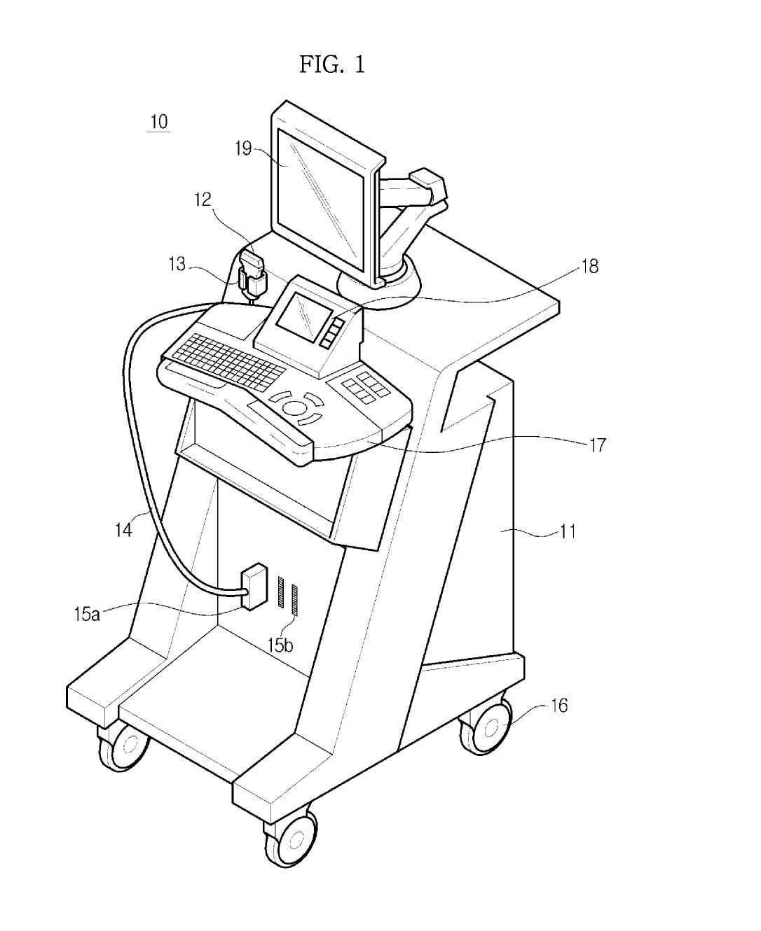

FIG. 1 is a perspective view illustrating an external appearance of a cart type ultrasonic diagnostic apparatus.

A cart type ultrasonic diagnostic apparatus, which is a high-end/premium ultrasonic diagnostic apparatus, has castors at the lower portion of a main body in order to overcome a disadvantage that it is inconvenient to move since the apparatus is large and heavy, although the apparatus has various functions.

Referring to FIG. 1, a cart type ultrasonic diagnostic apparatus 10 may include a main body 11 and an ultrasonic probe 12.

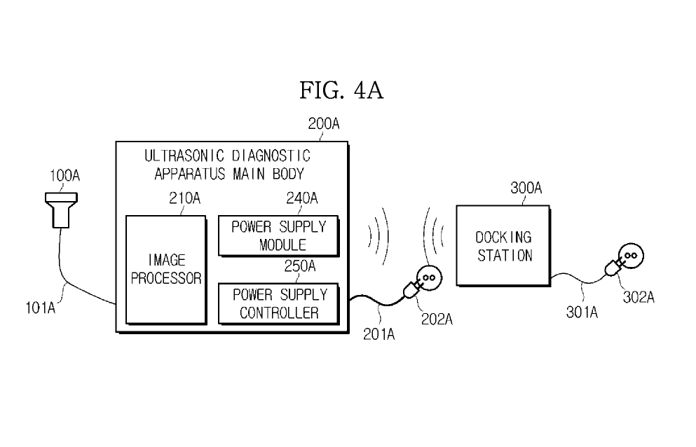

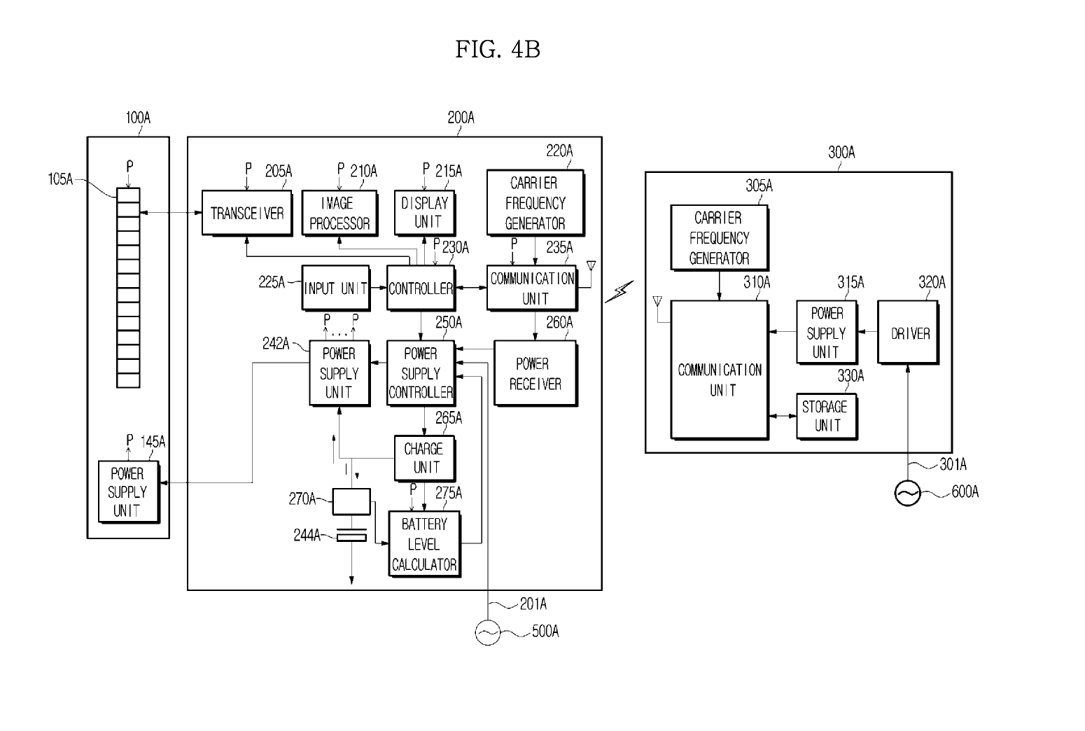

The main body 11 may accommodate main components, including, for example, a controller (see 230A of FIG. 4B) and an image processor (see 210A of FIG. 4B)) of the ultrasonic diagnostic apparatus 10. If an operator (that is, a user) inputs an ultrasonic diagnosis command, the controller may generate a transmission control signal and transmit the transmission control signal to the ultrasonic probe 12. Also, if an ultrasonic echo signal is received from the ultrasonic probe 12, the image processor (see 210A of FIG. 4B) may generate an ultrasonic image of a target region in an object based on the received ultrasonic echo signal.

In one side of the main body 11, one or more female connectors 15b may be provided. A male connector 15a connected to a cable 14 may be physically coupled with one of the female connectors 15b. A transmission signal generated by the controller may be transmitted to the ultrasonic probe 12 through the male connector 15a coupled with the female connector 15b of the main body 11 and the cable 14.

Meanwhile, in the lower portion of the main body 11 may be provided a plurality of castors 16 configured to move the ultrasonic diagnostic apparatus 10. The plurality of castors 16 can be used to fix the ultrasonic diagnostic apparatus 10 at a specific location, or the castors 16 may be used to move the ultrasonic diagnostic apparatus 10 in a specific direction.

The ultrasonic probe 12 may contact the body surface of an object (for example, a pregnant woman's abdomen) to transmit and receive ultrasonic waves. More specifically, the ultrasonic probe 12 may irradiate ultrasonic signals to an object based on a transmission signal received from the main body 11, receive ultrasonic echo signals reflected from a specific region (for example, the fetus) in the object, and transmit the ultrasonic echo signals to the main body 11.

To do this, in one end of the ultrasonic probe 12 may be provided a plurality of ultrasonic transducers configured to generate ultrasonic signals according to electrical signals.

Each ultrasonic transducer may generate ultrasonic waves according to applied alternating current power. More specifically, the ultrasonic transducer may receive alternating current power from an external power supply or an internal capacitor (for example, a battery), and a piezoelectric vibrator or a thin film of the ultrasonic transducer may vibrate according to the received alternating current power to generate ultrasonic waves.

The ultrasonic transducer may include any one or more of a magnetostrictive ultrasonic transducer using the magnetostrictive effect of a magnetic material, a piezoelectric ultrasonic transducer using the piezoelectric effect of a piezoelectric material, a capacitive micromachined ultrasonic transducer (CMUT) that transmits and receives ultrasonic waves using vibration of several hundreds or thousands of micromachined thin films, a Piezoelectric Micromachined Ultrasonic Transducer (pMUT), and/or a single crystal.

The ultrasonic transducers may be arranged in a linear array or in a convex array. Also, a cover for covering the ultrasonic transducers may be provided.

The other end of the ultrasonic probe 12 may be connected to one end of the cable 14, and the other end of the cable 14 may be connected to the male connector 15a. The male connector 15a may be physically coupled with the female connector 15b of the main body 11.

An input unit (also referred to herein as an “input device”) 17 enables a user to input commands related to operations of the ultrasonic diagnostic apparatus 10. For example, a user may use the input unit 17 to input any one or more of a mode selection command, a display command to display a combined mode consisting of two modes or more, a ultrasonic diagnosis start command, and so on, wherein modes for ultrasound images may include an Amplitude mode (A-mode), a Brightness mode (B-mode), a Color flow mode (C-mode), a Doppler mode (D-mode), a Power spectral mode (P-mode), and a Motion mode (M-mode).

The input unit 17 may include at least one of, for example, a touch pad, a keyboard, a foot switch, and a foot pedal. The touch pad or the keyboard may be implemented as hardware, and mounted on the upper portion of the main body 11. The keyboard may include at least one(s) of a switch, a key(s), a wheel, a joystick, a trackball, and a knob. As another example, the keyboard may be implemented as software, like a Graphic User Interface (GUI). In this case, the keyboard may be displayed on a sub display unit (also referred to herein as a “sub display device” and/or as a “sub display”) 18 or a main display unit (also referred to herein as a “main display device” and/or as a “main display”) 19. The foot switch or the foot pedal may be provided in the lower portion of the main body 11, and an operator may control operations of the ultrasonic diagnostic apparatus 10 by using the foot switch or the foot pedal.

A probe holder 13 for accommodating the ultrasonic probe 12 may be provided in relatively close proximity to the input unit 17. The operator may put the ultrasonic probe 12 into the probe holder 13 to safely keep the ultrasonic probe 12 when he/she does not use the ultrasonic diagnostic apparatus 10. In FIG. 1, one probe holder 13 is provided in proximity to the input unit 17, however, the probe holder 13 may be placed at a different location, or a plurality of probe holders may be provided, according to the entire design of the ultrasonic diagnostic apparatus 10 or according to the designs or locations of some components.

The sub display unit 18 may be mounted on the main body 11. In FIG. 1, the sub display unit 18 is provided over the input unit 17. The sub display unit 18 may include, for example, a Cathode Ray Tube (CRT) or a Liquid Crystal Display (LCD). The sub display unit 18 may display menus or guidance needed for ultrasonic diagnosis.

A main display unit 19 may be also mounted on the main body 11. In FIG. 1, the main display unit 19 is positioned over the sub display unit 18. The main display unit 19 may also include, for example, a CRT or a LCD. The main display unit 19 may display ultrasonic images acquired during ultrasonic diagnosis. Ultrasonic images that are displayed on the main display unit 19 may include at least one of a two-dimensional (2D) monochrome ultrasonic image, a 2D color ultrasonic image, a three-dimensional (3D) monochrome ultrasonic image, and a 3D color ultrasonic image.

In FIG. 1, the ultrasonic diagnostic apparatus 10 includes both the main display unit 19 and the sub display unit 18, however, the sub display unit 18 may be omitted, and in this case, applications or menus that are displayed through the sub display unit 18 may be displayed through the main display unit 19.

Also, at least one of the sub display unit 18 and the main display unit 19 may be removably connected to the main body 19.

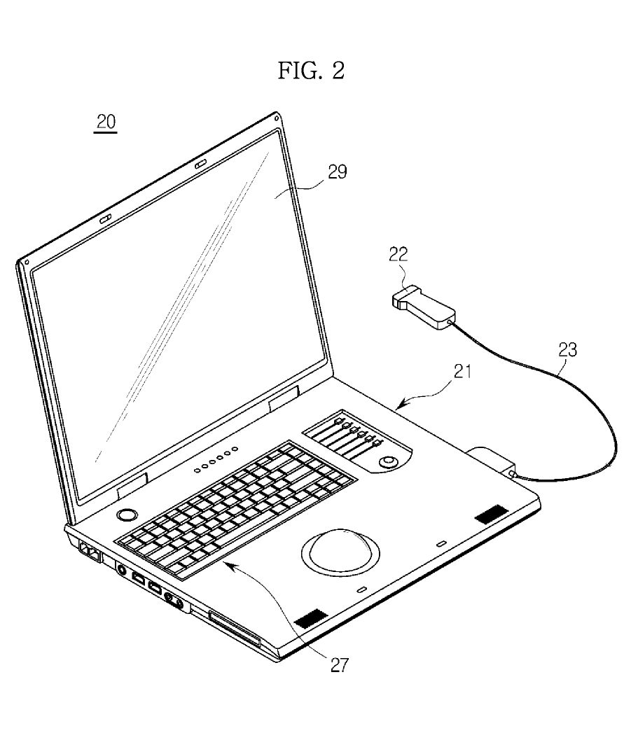

FIG. 2 is a perspective view illustrating an external appearance of a portable ultrasonic diagnostic apparatus.

The portable ultrasonic diagnostic apparatus is designed to be relatively compact and light-weight so that it can be easily moved in order to overcome a disadvantage of a conventional ultrasonic diagnostic apparatus that it is inconvenient to move since it is relatively large and heavy. Since the portable ultrasonic diagnostic apparatus can be easily moved, it can perform diagnosis regardless of place. Specifically, in FIG. 2, a portable ultrasonic diagnostic apparatus that is shaped like a laptop computer among various kinds of portable ultrasonic diagnostic apparatuses is shown.

As shown in FIG. 2, a portable ultrasonic diagnostic apparatus 20 may include a main body 21 and an ultrasonic probe 22.

The main body 21 may accommodate main components (for example, a controller (see 230A of FIG. 4B) and an image processor (see 210A of FIG. 4B)) of the portable ultrasonic diagnostic apparatus 20. If an operator (a user) inputs an ultrasonic diagnosis command, the controller may generate a transmission control signal, and transmit the transmission control signal to the ultrasonic probe 22. Also, if an ultrasonic echo signal is received from the ultrasonic probe 22, the image processor may create an ultrasonic image of a target region in an object based on the received ultrasonic echo signal. Also, a charge battery (e.g., a power battery) for driving the portable ultrasonic diagnostic apparatus 20 may be installed in the main body 21.

The ultrasonic probe 22 may be connected to one side of the main body 21 via a wired cable 23 or a wireless connection. The ultrasonic probe 22 may irradiate ultrasonic signals to an object based on a transmission control signal received from the controller in the main body 21, receive ultrasonic echo signals reflected from a target region in the object, and transmit the ultrasonic echo signals to the image processor in the main body 21.

Meanwhile, an input unit 27 mounted on the main body 21 may include a keyboard and a touch pad to perform functions of acquiring and controlling ultrasonic images, and a menu control function.

A display unit 29 which is foldably connected to the main body 21 may display ultrasonic images of an object, acquired by the image processor, and diagnosis information.

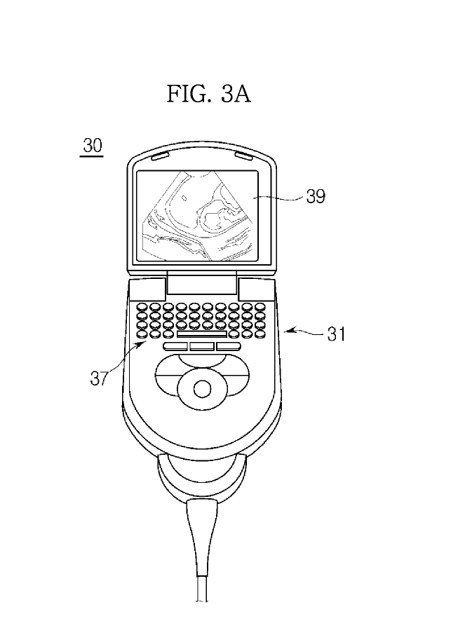

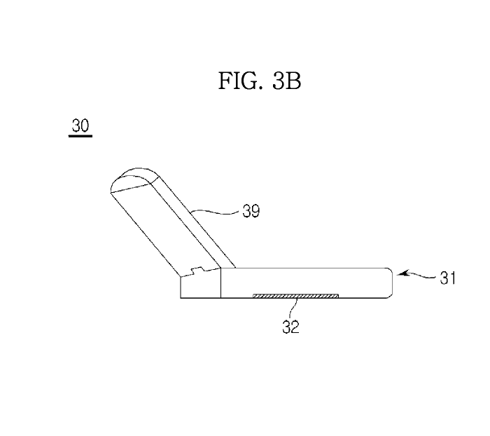

FIGS. 3A and 3B are views for describing an external structure of a handheld ultrasonic diagnostic apparatus.

Referring to FIGS. 3A and 3B, a handheld ultrasonic diagnostic apparatus 30, which is a kind of the portable ultrasonic diagnostic apparatus 20 as described above with reference to FIG. 2, is more compact and light-weight than the portable ultrasonic diagnostic apparatus 20 shown in FIG. 2. The handheld ultrasonic diagnostic apparatus 30 can be implemented as an ultrasonic probe. Like the portable ultrasonic diagnostic apparatus 20 shown in FIG. 2, in the handheld ultrasonic diagnostic apparatus 30, an ultrasonic probe or an ultrasonic probe handle may be connected to a main body (the main body is generally more compact than the main body 21 of the portable ultrasonic diagnostic apparatus 20 shown in FIG. 2) through a wired/wireless connection. Particularly, in FIGS. 3A and 3B, a handheld ultrasonic diagnostic apparatus that is shaped like a mobile phone, from among various kinds of handheld ultrasonic diagnostic apparatuses, is shown. In the following description, the handheld ultrasonic diagnostic apparatus 30 is also referred to as an ultrasonic probe or an ultrasonic probe handle.

As shown in FIGS. 3A and 3B, the ultrasonic probe 30 constituting the handheld ultrasonic diagnostic apparatus may include a casing 31 and a plurality of ultrasonic transducers 32.

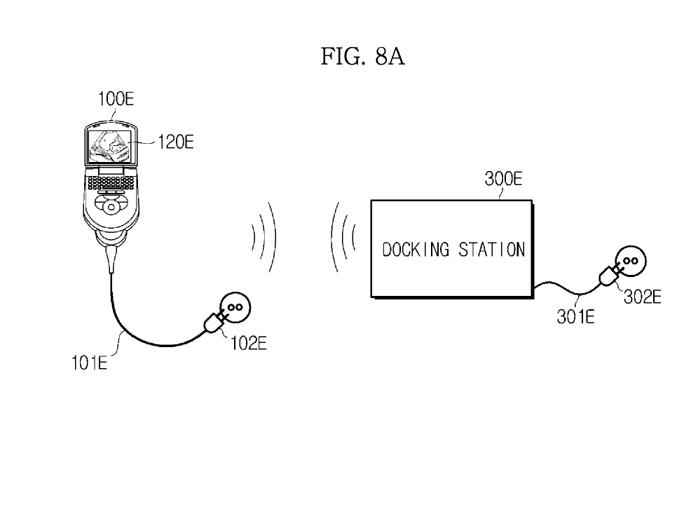

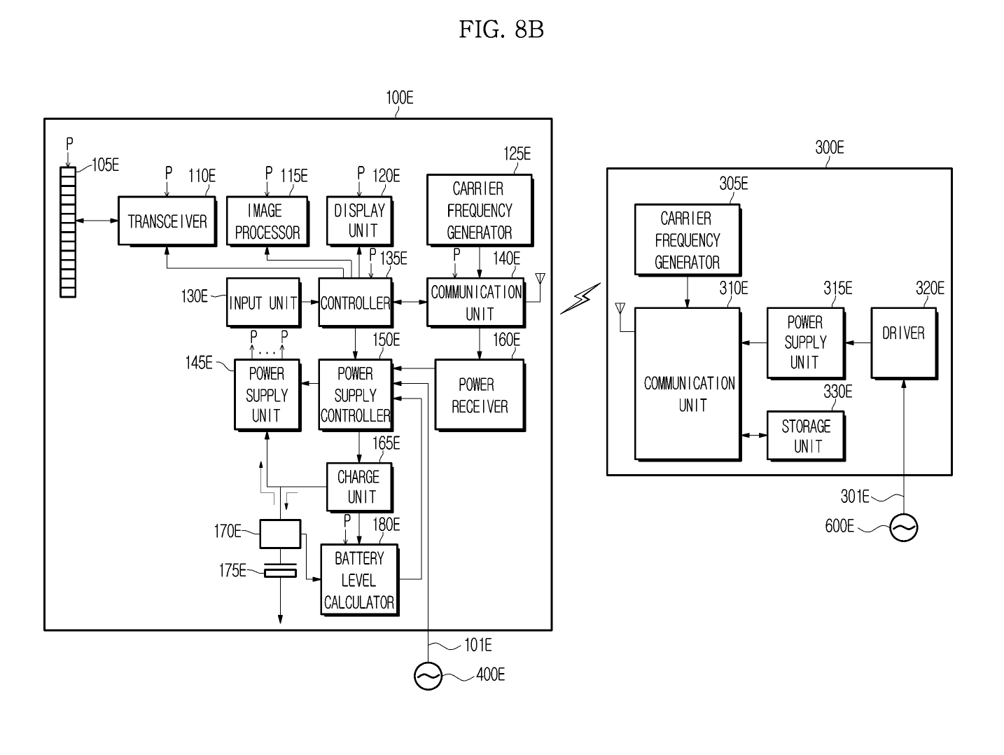

The casing 31 may form an outer appearance of the ultrasonic probe 30, and a controller (see 135E of FIG. 8B) and an image processor (see 115E of FIG. 8B) may be included in the casing 31. If an operator (a user) inputs an ultrasonic diagnosis command, the controller may generate a transmission control signal, and transmit the transmission control signal to the plurality of ultrasonic transducers 32. Further, if ultrasonic echo signals are received from the plurality of ultrasonic transducers 32, the image processor may generate an ultrasonic image of a target region in an object based on the received ultrasonic echo signals. In addition, a charge battery (a power battery) for driving the ultrasonic probe 30 may be installed in the casing 31.

The plurality of ultrasonic transducers 32 may be, as shown in FIG. 3B, arranged in the lower part of the casing 31. The plurality of ultrasonic transducers 32 may irradiate ultrasonic signals to an object based on a transmission control signal received from the controller included in the casing 31, receive ultrasonic echo signals reflected from a target region in the object, and transmit the ultrasonic echo signals to the image processor. The plurality of ultrasonic transducers 32 may be arranged in a linear array or in a convex array. In FIG. 3B, the plurality of ultrasonic transducers 32 are arranged in the lower part of the casing 31, however, it is also possible to attach an ultrasonic transducer module in which a plurality of ultrasonic transducers are arranged onto the lower or side part of the casing 31, and to scan the surface of an object using the ultrasonic transducer module connected to the casing 31 to transmit and receive ultrasonic signals.

Meanwhile, an input unit 37 mounted on the casing 31 may include a keyboard and a touch pad to perform functions of acquiring and controlling ultrasonic images, and a menu control function.

Further, a display unit 39 mounted on the casing 31 may display ultrasonic images of an object, formed by the image processor, and diagnosis information.

FIG. 4A is a control block diagram of an ultrasonic diagnostic system.

Referring to FIG. 4A, the ultrasonic diagnostic system may include an ultrasonic diagnostic apparatus including an ultrasonic probe 100A and an ultrasonic diagnostic apparatus main body 200A, and a docking station 300A.

The ultrasonic probe 100A may be connected to the ultrasonic diagnostic apparatus main body 200A through a wired cable 101A. The ultrasonic probe 100A may receive power and an ultrasonic transmission control signal from the ultrasonic diagnostic apparatus main body 200A through the wired cable 101A.

The ultrasonic diagnostic apparatus main body 200A may wirelessly receive power from the docking station 300A. Further, the ultrasonic diagnostic apparatus main body 200A may transmit ultrasonic information acquired from the ultrasonic probe 100A, and ultrasonic image information generated in the ultrasonic diagnostic apparatus main body 200A, to the docking station 300A, another ultrasonic diagnostic apparatus main body, or another electronic device, through wireless communication. Meanwhile, a detachable wired power cable 201A may be connected to the ultrasonic diagnostic apparatus main body 200A. The detachable wired power cable 201A is denoted by a thick solid line in FIG. 4A. One end of the detachable wired power cable 201A may be connected to a power plug 202A. The ultrasonic diagnostic apparatus main body 200A may receive power from an external commercial alternating current power source (see 500A of FIG. 4B) through the power plug 202A plugged in an electrical outlet. In particular, the ultrasonic diagnostic apparatus main body 200A may wirelessly receive power from the docking station 300A, or may receive power through the detachable wired power cable 201A.

The ultrasonic diagnostic apparatus main body 200A may include an image processor 210A which is configured to generate an ultrasonic image of a target region in an object based on ultrasonic echo signals received from the ultrasonic probe 100A, a power supply module 240A to supply power required from individual components in the ultrasonic diagnostic apparatus main body 200A, and a power supply controller 250A to control power that is supplied from external devices (the docking station 300A and the external commercial alternating current power source 500A). The power supply module 240A may include a power supply unit 242A and a charge battery 244A, which will be described below with reference to FIG. 4B.

The docking station 300A may wirelessly supply power to the ultrasonic diagnostic apparatus main body 200A, through a wireless power transfer technique. A wired power cable 301A may be connected to the docking station 300A, and one end of the wired power cable 301A may be connected to a power plug 302A. The docking station 300A may receive power from an external commercial alternating current power source (see 600A of FIG. 4B) through the power plug 302A plugged in an electrical outlet, and supply the received power to the ultrasonic diagnostic apparatus main body 200A through the wireless power transfer technique.

FIG. 4B is a control block diagram illustrating configurations of the ultrasonic probe 100A, the ultrasonic diagnostic apparatus main body 200A, and the docking system 300A shown in FIG. 4A.

Referring to FIG. 4B, the ultrasonic probe 100A may include an ultrasonic transducer array 105A, and may further include a power supply unit (also referred to herein as a “power supply”) 145A.

The ultrasonic transducer array 105A may include an array of a plurality of ultrasonic transducers, and the plurality of ultrasonic transducers may be arranged in a linear array or in a convex array, as described above with reference to FIG. 4A. Each ultrasonic transducer may include any one or more of a magnetostrictive ultrasonic transducer using the magnetostrictive effect of a magnetic material, a piezoelectric ultrasonic transducer using the piezoelectric effect of a piezoelectric material, a capacitive micromachined ultrasonic transducer (CMUT) that transmits and receives ultrasonic waves using vibration of several hundreds or thousands of micromachined thin films, a Piezoelectric Micromachined Ultrasonic Transducer (pMUT), and/or a single crystal.

The power supply unit 145A may convert power received from the ultrasonic diagnostic apparatus main body 200A through the wired cable 101A (see FIG. 4A), into a form of power that can be appropriately used by the ultrasonic transducer array 105A, and supply the converted power to the ultrasonic transducer array 105A.

As shown in FIG. 4B, the ultrasonic diagnostic apparatus main body 200A may include a transceiver 205A. The ultrasonic transducer array 105A in the ultrasonic probe 100A may be connected to the transceiver 205A in the ultrasonic diagnostic apparatus main body 200A through the wired cable 101A. In particular, the ultrasonic probe 100A may receive power from the ultrasonic diagnostic apparatus main body 200A through the wired cable 101A, or may transmit/receive various information (ultrasonic signals, control signals, etc.) to/from the ultrasonic diagnostic apparatus main body 200A through the wired cable 101A. The transceiver 205A may be a device which includes electronic circuits capable of transmitting/receiving ultrasonic signals, such as any one or more of a Low Noise Amplifier (LNA), a Variable Gain Amplifier (VGA), an Analog-to-Digital Converter (ADC), a switch, a multiplexer (MUX), a transmit beamformer, a receive beamformer, a pulser, a pulser driver, etc. The transceiver 205A can be defined as a front-end module. The transceiver 205A may transmit a driving signal to the ultrasonic transducer array 105A in order for the ultrasonic transducer array 105A to transmit ultrasonic waves to a target region in an object. Further, the transceiver 205A may receive ultrasonic echo signals reflected from the target region in the object through the ultrasonic transducer array 105A. The transceiver 205A may be electrically connected to the controller 230A. The transceiver 205A may transmit/receive ultrasonic waves based on an ultrasonic transmission/reception control signal received from the controller 230A. In addition, the transceiver 205A may transfer ultrasonic echo signals received from the ultrasonic transducer array 105A to the image processor 210A.

The image processor 210A may receive the ultrasonic echo signals from the transceiver 205A, and generate an ultrasonic image (or diagnosis information) of the target region in the object, based on the ultrasonic echo signals. The diagnosis information may include, for example, any one or more of a B-mode image, a Color flow image, and/or a Doppler spectrum image. The B-mode image is a section image of the object to be diagnosed, the Color flow image is an image of blood flow or blood velocity distribution with respect to the object to be diagnosed, and the Doppler spectrum image represents the velocity and direction of blood flow using the spectrum of Doppler signals. Various diagnosis information (for example, an ultrasonic image) which relates to the object, generated by the image processor 210A, may be displayed on a display unit 215A connected to the image processor 210A.

The image processor 210A and the display unit 215A may be controlled by the controller 230A. Further, the controller 230A may transmit an ultrasonic transmission/reception control signal to the transceiver 205A. An input unit 225A may be electrically connected to the controller 230A. The input unit 225A enables an operator (a user) to input various commands, such as a mode setting command (for example, a wireless power transfer mode setting command) and an ultrasonic diagnosis start command, or various types of information related to operations of the ultrasonic diagnostic apparatus.

The controller 230A may be electrically connected to a communication unit (also referred to herein as a “communicator”) 235A. The controller 230A may transmit various information, such as ultrasonic echo signals received from the transceiver 205A and an ultrasonic image (diagnosis information) of an object, received from the image processor 210A, to the docking station 300A, through the communication unit 235A.

The communication unit 235A may be used for wireless communication or radio communication. For example, the communication unit 235A may transmit/receive various information, such as ultrasonic echo signals and ultrasonic images (diagnosis information), to/from the docking station 300A, wirelessly, using electronic waves (wireless data communication). However, the communication unit 235A may communicate with the docking station 300A using light, instead of electronic waves, wherein the light may be visible light or invisible light. The communication unit 235A may wirelessly transmit various information, such as the ultrasonic echo signals and the ultrasonic images (diagnosis information), to the docking station 300A, by using a carrier frequency generated by a carrier frequency generator 220A. An antenna for transmitting or receiving electronic wave energy may be connected to the communication unit 235A.

Further, the communication unit 235A may wirelessly receive power from the docking station 300A (i.e., via a wireless power transfer). The wireless power transfer is a non-contact-based system of transferring power without any contact between a power source and an electronic device, and may be implemented through any one or more of inductive coupling, resonant magnetic coupling, RF-based wireless power, or the like. The communication unit 235A may transfer power received from the docking station 300A to a power receiver 260A.

The power receiver 260A may receive power supplied through the wireless power transfer technique. The power receiver 260A may receive power supplied wirelessly through any one or more of a capacitive method using an electric field, a resonance method using a magnetic field, or an inductive method, and transfer the received power to a power supply controller 250A.

The power supply controller 250A is a circuitry which is configured for controlling power that is supplied from external devices (the docking station 300A and an external commercial alternating current power source 500A). The power supply controller 250A may be implemented as a switch. If the power supply controller 250A receives power from the external commercial alternating current source 500A through the detachable wired power cable 201A, the power supply controller 250A may transfer the received power to a power supply unit 242A. Then, the power supply unit 242A may convert the power received from the power supply controller 250A into a form of power that can be appropriately used to operate each of individual components (for example, the transceiver 205A, the image processor 210A, the display unit 215A, the controller 230A, etc.) in the ultrasonic diagnostic apparatus main body 200A, and then supply the converted power to the corresponding component. In addition, the power supply unit 242A may transfer power needed to drive the ultrasonic transducer array 105A in the ultrasonic probe 100A, to the power supply unit 145A in the ultrasonic probe 100A, through the wired cable 101A.

Meanwhile, if the power supply controller 250A receives power from the power receiver 260A, the power supply controller 250A may transfer the received power to a charge unit (also referred to herein as a “charger”) 265A. Then, a charge battery 244A may be charged by the charge unit 265A. The charge unit 265A may charge power received from the power receiver 260A and the power supply controller 250A in the charge battery 244A. The charge unit 265A may charge the power in the charge battery 244A through any one or more of the capacitive method using the electric field, the resonance method using the magnetic field, and/or the inductive method. The power supply unit 242A may convert power accumulated in the charge battery 244A into a form of power that can be appropriately used to operate each of the individual components (for example, the transceiver 205A, the image processor 210A, the display unit 215A, the controller 230A, etc.) in the ultrasonic diagnostic apparatus main body 200A, and supply the converted power to the corresponding component.

The charge battery 244A may include a primary battery and/or a secondary battery. If the charge battery 244A is a secondary battery, it is possible to separate the charge battery 244A from the ultrasonic diagnostic apparatus main body 200A and then to charge power in the charge battery 244A.

A current sensor 270A may be connected in series to the charge battery 244A. The current sensor 270A may detect an amount and direction of current. Information detected by the current sensor 270A may be transferred to a battery level calculator 275A. The battery level calculator 275A may accumulatively add current entering the charge battery 244A over time in order to calculate a charge amount, accumulatively add current discharged from the charge battery 244A over time in order to calculate a discharge amount, and then calculate a battery level of the charge battery 244A based on a difference between the charge amount and the discharge amount. The battery level of the charge battery 244A calculated by the battery level calculator 275A may be displayed on the display unit 215A. The display unit 215A may display, in addition to the battery level of the charge battery 244A, any one or more of a wireless communication state (for example, transmission stable or unstable), a current mode (for example, an ultrasonic transmission/reception mode, an ultrasonic non-transmission/reception mode, and/or a wireless power transfer mode) of the ultrasonic diagnostic system, etc. An operator (a user) may check the charge state (the battery level) of the charge battery 244A, displayed on the display unit 215A, and set the wireless power transfer mode through the input unit 225A. If the controller 230A receives a wireless power transfer mode setting command from the input unit 225A, the controller 230A may control the communication unit 235A and the power supply controller 250A to receive power from the docking station 300A through the wireless power transfer technique, and to charge the power in the charge battery 244A.

As shown in FIG. 4B, the docking station 300A may include a power supply unit 315A. The power supply unit 315A is used to supply power to the power receiver 260A in the ultrasonic diagnostic apparatus main body 200A through the inductive method. The power supply unit 315A may be driven by a driver 320A. The driver 320A may be connected to an external commercial alternating current power source 600A through the wired power cable 301A. The driver 320A may transfer power received from the external commercial alternating current power source 600A to the power supply unit 315A.

Meanwhile, the power supply unit 315A may be electrically connected to a communication unit 310A. The power supply unit 315A may transfer the power received from the driver 320A to the communication unit 315A.

The communication unit 310A is used for wireless communication or radio communication. For example, the communication unit 310A may transfer power to the ultrasonic diagnostic apparatus main body 200A, wirelessly (wireless power transfer). The wireless power transfer is a non-contact-based system of transferring power without any contact between a power source and an electronic device, and may be implemented through any one or more of inductive coupling, resonant magnetic coupling, RF-based wireless power, and/or the like. The communication unit 310A may wirelessly transmit power received from the external commercial alternating current power source 600A to the ultrasonic diagnostic apparatus main body 200A, by using a carrier frequency generated by a carrier frequency generator 305A. An antenna configured for transmitting or receiving electronic wave energy may be connected to the communication unit 310A.

Further, the communication unit 310A may wirelessly transmit/receive ultrasonic echo signals or ultrasonic images (diagnosis information) to/from the ultrasonic diagnostic apparatus main body 200A, using electronic waves (i.e., via wireless data communication). However, the communication unit 310A may communicate with the ultrasonic diagnostic apparatus main body 200A using light, instead of electronic waves, wherein the light may be visible light or invisible light. Various information, such as ultrasonic echo signals and ultrasonic images (diagnosis information), transmitted wirelessly from the ultrasonic diagnostic apparatus main body 200A through the communication unit 310A, may be stored in a storage unit (also referred to herein as a “storage device” and/or as a “storage”) 330A.

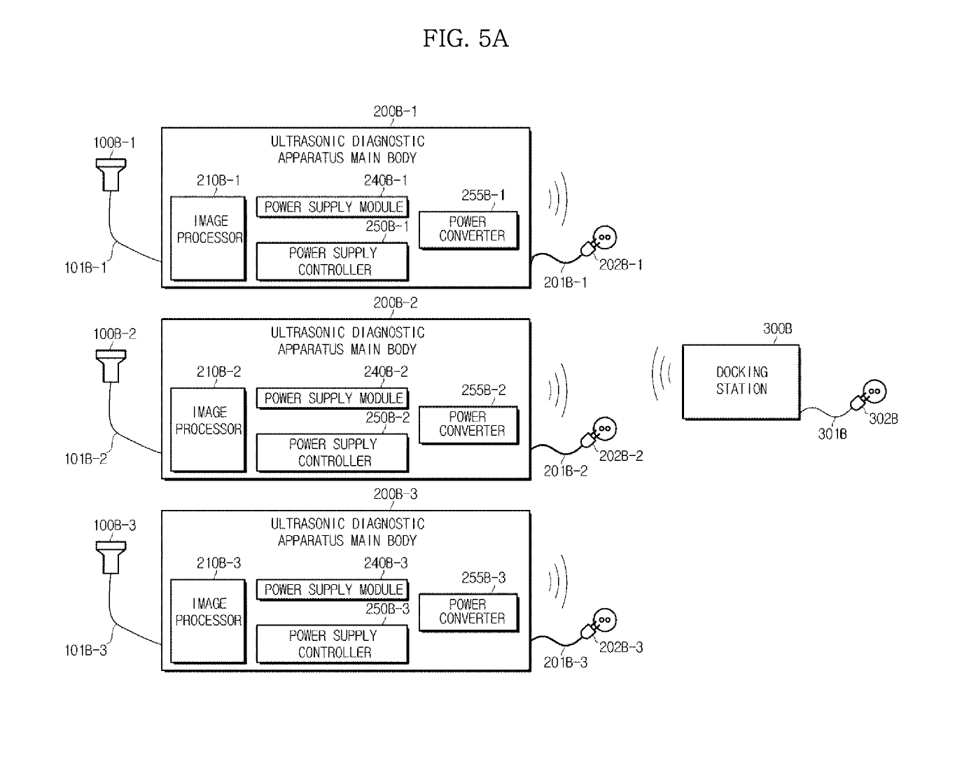

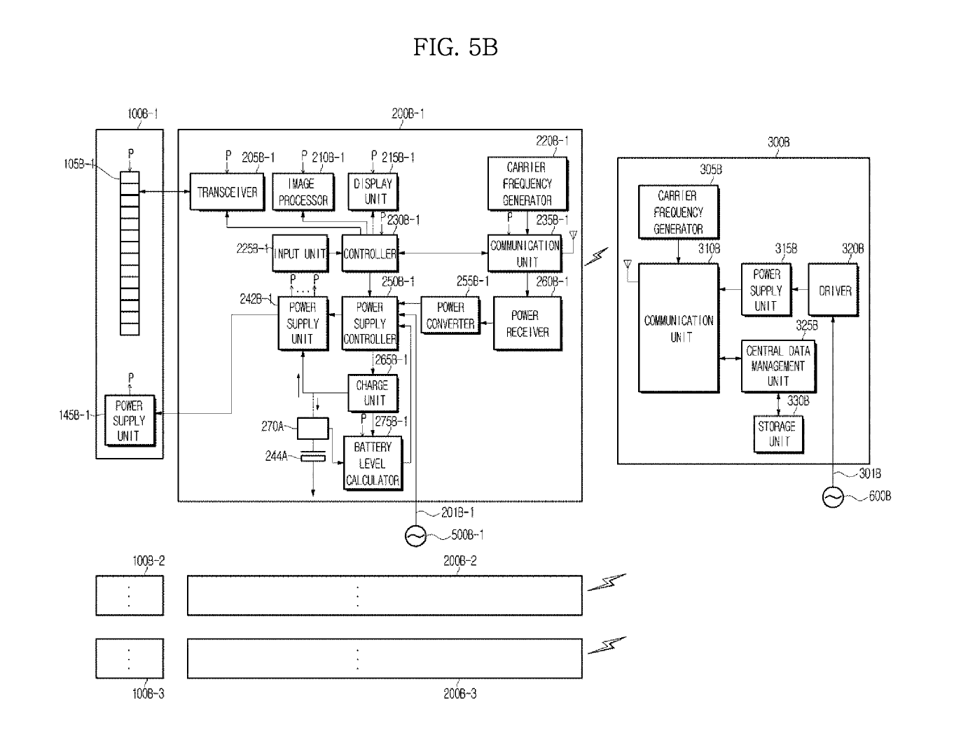

FIG. 5A is a control block diagram of an ultrasonic diagnostic system.

The above description given with reference to FIGS. 4A and 4B relates to a control configuration of an ultrasonic diagnostic system according to an exemplary embodiment. In FIGS. 4A and 4B, a system in which the ultrasonic diagnostic apparatus main body 200A receives power wirelessly from the docking station 300A is shown, whereas in FIG. 5A, a system in which a plurality of ultrasonic diagnostic apparatus main bodies 200B-1 200B-2, and 200B-3 receive power wirelessly from a docking station 300B is shown.

As shown in FIG. 5A, an ultrasonic diagnostic system may include a plurality of ultrasonic diagnostic apparatuses which include of a plurality of ultrasonic probes 100B-1, 100B-2, and 100B-3 and a plurality of ultrasonic diagnostic apparatus main bodies 200B-1, 200B-2, and 200B-3, respectively, and a docking station 300B.

The respective ultrasonic probes 100B-1, 100B-2, and 100B-3 may be connected to the respective ultrasonic diagnostic apparatus main bodies 200B-1, 200B-2, and 200B-3 through a plurality of wired cables 101B-1, 101B-2, and 101B-3, respectively. The respective ultrasonic probes 100B-1, 100B-2, and 100B-3 may receive power and ultrasonic transmission control signals from the respective ultrasonic diagnostic apparatus main bodies 200B-1, 200B-2, and 200B-3 through the respective wired cables 101B-1, 101B-2, and 101B-3.

The respective ultrasonic diagnostic apparatus main bodies 200B-1, 200B-2, and 200B-3 may receive power from the docking station 300B. In addition, the respective ultrasonic diagnostic apparatus main bodies 200B-1, 200B-2, and 200B-3 may transmit ultrasonic information acquired from the respective ultrasonic probes 100B-1, 100B-2, and 100B-3 through wireless communication, and ultrasonic image information generated by the respective ultrasonic diagnostic apparatus main bodies 200B-1, 200B-2, and 200B-3, to the docking station 300B. Meanwhile, a plurality of detachable wired power cables 201B-1, 201B-2, and 201B-3 may be connected to the respective ultrasonic diagnostic apparatus main bodies 200B-1, 200B-2, and 200B-3. One ends of the detachable wired power cables 201B-1, 201B-2, and 201B-3 may be connected to a plurality of power plugs 202B-1, 202B-2, and 202B-3, respectively. The respective ultrasonic diagnostic apparatus main bodies 200B-1, 200B-2, and 200B-3 may receive power from external commercial alternating current power sources (see 500B-1 of FIG. 5B) through the respective power plugs 202B-1, 202B-2, and 202B-3 plugged in electrical outlets. In particular, the respective ultrasonic diagnostic apparatus main bodies 200B-1, 200B-2, and 200B-3 may wirelessly receive power from the docking station 300B, or receive power through the detachable wired power cables 201B-1, 201B-2, and 201B-3.

The respective ultrasonic diagnostic apparatus main bodies 200B-1, 200B-2, and 200B-3 may include a plurality of image processors 210B-1, 210B-2, and 210B-3 which are configured to generate an ultrasonic image for a target region in an object based on ultrasonic echo signals received from the respective ultrasonic probes 100B-1, 100B-2, and 100B-3, a plurality of power supply modules 240B-1, 240B-2, and 240B-3 configured to supply needed power to individual components in the respective ultrasonic diagnostic apparatus main bodies 200B-1, 200B-2, and 200B-3, a plurality of power supply controllers 250B-1, 250B-2, and 250B-3 configured to control power received from external devices (the docking station 300A and the external commercial alternating current power sources 500B-1), and a plurality of power converters 255B-1, 255B-2, and 255B-3 configured to convert power received from the docking station 300B into a form of power that can be appropriately used by the respective ultrasonic diagnostic apparatus main bodies 200B-1, 200B-2, and 200B-3. Each of the power supply modules 240B-1, 240B-2, and 240B-3 may include a power supply unit 242B-1 and a charge battery 244B-1, as shown in FIG. 5B.

The docking station 300B may wirelessly supply power to the respective ultrasonic diagnostic apparatus main bodies 200B-1, 200B-2, and 200B-3, through the wireless power transfer technique. A wired power cable 301B may be connected to the docking station 300B, and one end of the wired power cable 301B may be connected to a power plug 302B. The docking station 300B may receive power from an external commercial alternating current power source (see 600B of FIG. 5B) through the power plug 302B plugged in an electrical outlet, and supply the received power to the respective ultrasonic diagnostic apparatus main bodies 200B-1, 200B-2, and 200B-3 through the wireless power transfer technique.

FIG. 5B is a control block diagram illustrating configurations of the ultrasonic probes 100B-1, 100B-2, and 100B-3, the ultrasonic diagnostic apparatus main bodies 200B-1, 200B-2, and 200B-3, and the docking system 300B shown in FIG. 5A.

Since the ultrasonic probes 100B-1, 100B-2, and 100B-3 have the same configuration, and the ultrasonic diagnostic apparatus main bodies 200B-1, 200B-2, and 200B-3 also have the same configuration, in FIG. 5B, only configurations of the first ultrasonic probe 100B-1 and the first ultrasonic diagnostic apparatus main body 200B-1 are shown in detail, and configurations of the second and third ultrasonic probes 100B-2 and 100B-3 and the second and third ultrasonic diagnostic apparatus main bodies 200B-2 and 200B-3 are not shown.

In addition, the configuration of each of the ultrasonic diagnostic apparatus main bodies 200B-1, 200B-2, and 200B-3 as shown in FIGS. 5A and 5B are the same as the configuration of the ultrasonic diagnostic apparatus main body 200A shown in FIGS. 4A and 4B, except that the ultrasonic diagnostic apparatus main bodies 200B-1, 200B-2, and 200B-3 further include a plurality of power converters 255B-1, 255B-2, and 255B-3 configured to convert power supplied from the docking station 300B into a form of power that can be appropriately used by the respective ultrasonic diagnostic apparatus main bodies 200B-1, 200B-2, and 200B-3. Accordingly, detailed descriptions for the individual components in the ultrasonic diagnostic apparatus main bodies 200B-1, 200B-2, and 200B-3 will be omitted. Further, since the configuration of each of the ultrasonic probes 100B-1, 100B-2, and 100B-3 shown in FIGS. 5A and 5B is also the same as the configuration of the ultrasonic probe 100A shown in FIGS. 4A and 4B, detailed descriptions for the individual components in the ultrasonic probes 100B-1, 100B-2, and 100B-3 will be omitted.

As shown in FIG. 5B, the docking station 300B may include a power supply unit 315B. The power supply unit 315B may supply power to a power receiver 260B in each of the ultrasonic diagnostic apparatus main bodies 200B-1, 200B-2, and 200B-3 through the inductive method or the like. The power supply unit 315B may be driven by a driver 320B. The driver 320B may be connected to the external commercial alternating current power source 600B through the wired power cable 301B. The driver 320B may transfer power received from the external commercial alternating current power source 600B to the power supply unit 315B.

Meanwhile, the power supply unit 315B may be electrically connected to the communication unit 310B. The power supply unit 315B may transfer power received from the driver 320B to the communication unit 310B.

The communication unit 310B is used for wireless communication or radio communication. For example, the communication unit 310B may wirelessly transmit power to the ultrasonic diagnostic apparatus main bodies 200B-1, 200B-2, and 200B-3 (i.e., via a wireless power transfer). The wireless power transfer is a non-contact-based system of transferring power without any physical contact between a power source and an electronic device, and may be implemented through any one or more of inductive coupling, resonant magnetic coupling, RF-based wireless power, or the like. The communication unit 310B may transmit power received from the external commercial alternating current power source 600B to the ultrasonic diagnostic apparatus main bodies 200B-1, 200B-2, and 200B-3, wirelessly, using a carrier frequency generated by a carrier frequency generator 305A. An antenna for transmitting or receiving electronic wave energy may be connected to the communication unit 310B.

Further, the communication unit 310B may wirelessly transmit/receive various information, such as ultrasonic echo signals and ultrasonic images (diagnosis information), to/from the ultrasonic diagnostic apparatus main bodies 200B-1, 200B-2, and 200B-3, by using electronic waves (wireless data communication). However, the communication unit 310B may communicate with the ultrasonic diagnostic apparatus main bodies 200B-1, 200B-2, and 200B-3 using light, instead of electronic waves, wherein the light may be visible light or invisible light. The various information, such as the ultrasonic echo signals and the ultrasonic images (diagnosis information), transmitted wirelessly from the respective ultrasonic diagnostic apparatus main bodies 200B-1, 200B-2, and 200B-3 through the communication unit 310B may be transferred to a central data management unit (also referred to herein as a “central management device” and/or as a “central manager”) 325B.

The central data management unit 325B may manage the various information transmitted wirelessly from the respective ultrasonic diagnostic apparatus main bodies 200B-1, 200B-2, and 200B-3. The central data management unit 325B may store information that needs to be stored, from among the various information, in a storage unit 330B. Further, the central data management unit 325B may read, when receiving a data transmission request from each ultrasonic diagnostic apparatus main body 200B-1, 200B-2, or 200B-3, the various information stored in the storage unit 330B, and wirelessly transmit the read information to the corresponding ultrasonic diagnostic apparatus main body 200B-1, 200B-2, or 200B-3 through the communication unit 310B.

As shown in FIGS. 5A and 5B, in the ultrasonic diagnosis system that the plurality of ultrasonic diagnostic apparatuses in which the plurality of ultrasonic probes 100B-1, 100B-2, and 100B-3 and the ultrasonic diagnostic apparatus main bodies 200B-1, 200B-2, and 200B-3 receive power wirelessly from the docking station 300B, the docking station 300B may function as a hub of power supply. In addition, in the ultrasonic diagnosis system that the plurality of ultrasonic diagnostic apparatuses in which the plurality of ultrasonic probes 100B-1, 100B-2, and 100B-3 and the ultrasonic diagnostic apparatus main bodies 200B-1, 200B-2, and 200B-3 can wirelessly transmit/receive data to/from the docking station 300B, the docking station 300B may function as a data hub.

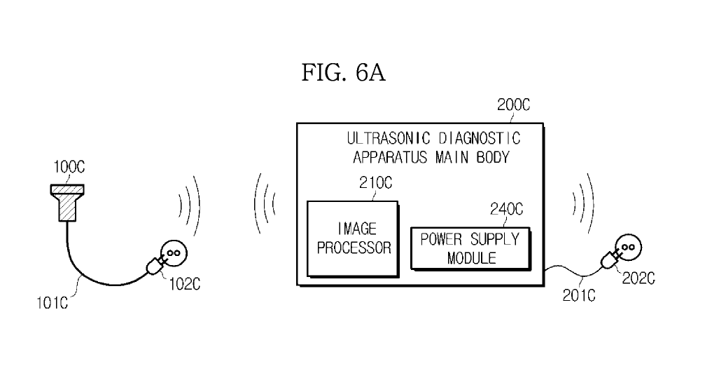

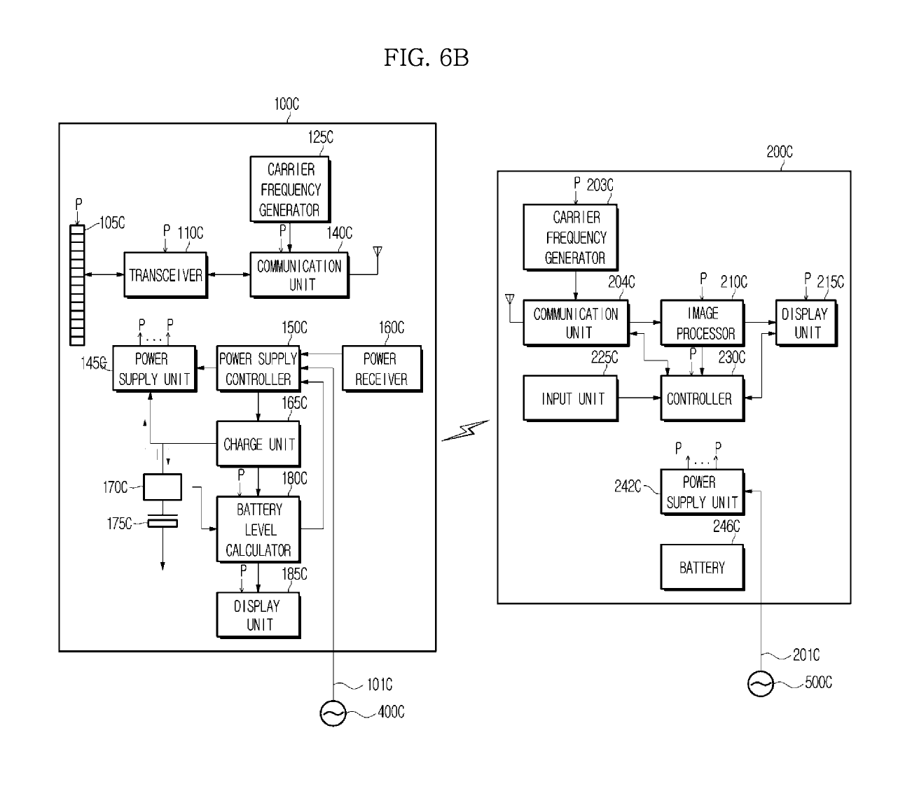

FIG. 6A is a control block diagram of an ultrasonic diagnostic apparatus.

In the above-described exemplary embodiments, an ultrasonic diagnostic system in which one or more ultrasonic diagnostic apparatuses can receive power from a docking station wirelessly has been described. Hereinafter, an ultrasonic diagnostic apparatus in which a ultrasonic probe can receive power from an ultrasonic diagnostic apparatus main body wirelessly will be described in detail with reference to FIGS. 6A and 6B.

As shown in FIG. 6A, an ultrasonic diagnostic apparatus may include an ultrasonic probe 100C and an ultrasonic diagnostic apparatus main body 200C.

The ultrasonic probe 100C may wirelessly receive power from the ultrasonic diagnostic apparatus main body 200C. Further, the ultrasonic probe 100C may transmit ultrasonic information which relates to an object, acquired by an ultrasonic transducer array (see 105C of FIG. 6B), to the ultrasonic diagnostic apparatus main body 200C, through wireless communication. Meanwhile, a detachable wired power cable 101C may be connected to the ultrasonic probe 100C. One end of the detachable wired power cable 101C may be connected to a power plug 102C. The ultrasonic probe 100C may receive power from an external commercial alternating current power source (see 400C of FIG. 6B) through the power plug 102C plugged in an electrical outlet. In particular, the ultrasonic probe 100C may wirelessly receive power from the ultrasonic diagnostic apparatus main body 200C, or may receive power through the detachable wired power cable 101C.

The ultrasonic diagnostic apparatus main body 200C may wirelessly supply power to the ultrasonic probe 100C, through the wireless power transfer technique. A wired power cable 201C may be connected to the ultrasonic diagnostic apparatus main body 200C, and one end of the wired power cable 201C may be connected to a power plug 202C. The ultrasonic diagnostic apparatus main body 200C may receive power from an external commercial alternating current power source (see 500C of FIG. 6B) through the power plug 202C plugged in an electrical outlet, and supply the received power to the ultrasonic probe 100C.

The ultrasonic diagnostic apparatus main body 200C may include an image processor 210C which is configured to generate an ultrasonic image of a target region in an object based on ultrasonic echo signals received from the ultrasonic probe 100C, and a power supply module 240C configured to supply needed power to individual components in the ultrasonic diagnostic apparatus main body 200C. The power supply module 240C may include a power supply unit 242C and a battery 246C, which will be described below with reference to FIG. 6B.

FIG. 6B is a control block diagram illustrating configurations of the ultrasonic probe 100C and the ultrasonic diagnostic apparatus main body 200C shown in FIG. 6A.

As shown in FIG. 6B, the ultrasonic probe 100C may include an ultrasonic transducer array 105C in which a plurality of ultrasonic transducers are arranged in an array.

The ultrasonic transducer array 105C may be electrically connected to a transceiver 110C. The transceiver 110C may transmit a driving signal to the ultrasonic transducer array 105C in order for the ultrasonic transducer array 105C to irradiate ultrasonic waves to a target region in an object. Further, the transceiver 110C may receive ultrasonic echo signals reflected from the target region in the object from the ultrasonic transducer array 105C. The transceiver 110C may be connected to a communication unit 140C. The transceiver 110C may transmit and receive ultrasonic waves, based on an ultrasonic transmission/reception control signal received from the ultrasonic diagnostic apparatus main body 200C through the communication unit 140C. In addition, the transceiver 110C may transmit ultrasonic echo signals reflected from the target region in the object, transferred from the ultrasonic transducer array 105C, to the ultrasonic diagnostic apparatus main body 200C, through the communication unit 140C.

The communication unit 140C is used for wireless communication. For example, the communication unit 140C may wirelessly transmit/receive various information, such as ultrasonic echo signals and an ultrasonic transmission/reception signal, to/from the ultrasonic diagnostic apparatus main body 200C, by using electronic waves (i.e. wireless data communication). However, the communication unit 140C may communicate with the ultrasonic diagnostic apparatus main body 200C, using light, instead of electronic waves, wherein the light may be visible light or invisible light. The communication unit 140C may transmit ultrasonic information (ultrasonic echo signals) for the object, to the ultrasonic diagnostic apparatus main body 200C, wirelessly, using a carrier frequency generated by a carrier frequency generator 125C. An antenna for transmitting or receiving electronic wave energy may be connected to the communication unit 140C.

Further, the communication unit 140C may receive power from the ultrasonic diagnostic apparatus main body 200C, wirelessly (wireless power transfer). The wireless power transfer is a non-contact-based system of transferring power without any contact between a power source and an electronic device, and may be implemented through any one or more of inductive coupling, resonant magnetic coupling, RF-based wireless power, and/or the like. The communication unit 140C may transfer power received from the ultrasonic diagnostic apparatus main body 200C to a power receiver 160C.

At this time, an arbitrary frequency in an ultrasonic frequency band may be set to a carrier frequency for wireless data communication or wireless power transfer. In this case, in an ultrasonic non-transmission/reception mode (for example, a freeze mode), wireless data communication or wireless power transfer may be performed by using ultrasonic pulses generated from the ultrasonic transducer array 105C. In the case in which an arbitrary frequency in an ultrasonic frequency band is set to a carrier frequency for wireless data communication or wireless power transfer, the carrier frequency generator 125C may be omitted.

The power receiver 160C may receive power supplied through the wireless power transfer technique. The power receiver 160C may receive power supplied wirelessly through the inductive method or the like, and transfer the received power to the power supply controller 150C.

The power supply controller 150C may control power supplied from external devices (the ultrasonic diagnostic apparatus main body 200C and the external commercial alternating current power source 400C). For example, the power supply controller 150C may be implemented as a switch. If the power supply controller 150C receives power from the external commercial alternating current power source 400C through the detachable wired power cable 101C, the power supply controller 150C may transfer the received power to the power supply unit 145C. The power supply unit 145C may convert the power received through the power supply controller 150C into a form of power that can be appropriately used to operate each of individual components (for example, the ultrasonic transducer array 105C, the transceiver 110C, the communication unit 140C, a battery level calculator 180C, a display unit 185C, etc.) in the ultrasonic probe 100C, and transfer the converted power to the corresponding component.

Meanwhile, if the power supply controller 150C receives power from the power receiver 160C, the power supply controller 150C may transfer the received power to a charge unit 165C. A charge battery 175C may be charged by the charge unit 165C. The charge unit 165C may charge power received through the power receiver 160C and the power supply controller 150C in the charge battery 175C. If the power supply controller 150C receives a wireless power transfer mode setting command from an operator (a user) through an input unit 225C of the ultrasonic diagnostic apparatus main body 200C, the power supply controller 150C may enter a wireless power transfer mode to charge power supplied from the power receiver 160C in the charge battery 175C, or in an ultrasonic non-transmission/reception mode (for example, a freeze mode), the power supply controller 150C may be automatically switched to the wireless power transfer mode (automatic mode switching) to charge power supplied from the power receiver 160C in the charge battery 175C. The charge battery 175C may be charged through any one or more of a capacitive method using an electric field, a resonance method using a magnetic field, and/or an inductive method. The power supply unit 145C may convert power that is accumulated in the charge battery 175C into a form of power that can be appropriately used to operate each of the individual components (for example, the ultrasonic transducer array 105C, the transceiver 110C, the communication unit 140C, the battery level calculator 180C, the display unit 185C, etc.) in the ultrasonic probe 100C, and supply the converted power to the corresponding component.

The charge battery 175C may be a primary battery or a secondary battery. If the charge battery 175C is a secondary battery, it is possible to separate the charge battery 175C from the ultrasonic probe 100C and then charge power in the charge battery 175C.

A current sensor 170C may be connected in series to the charge battery 175C. The current sensor 170C may detect an amount and direction of current. Information detected by the current sensor 175C may be transferred to the battery level calculator 180C. The battery level calculator 180C may accumulatively add current entering the charge battery 175C over time to calculate a charge amount, accumulatively add current discharged from the charge battery 175C over time to calculate a discharge amount, and then calculate a battery level of the charge battery 175C based on a difference between the charge amount and the discharge amount. The battery level of the charge battery 175C, calculated by the battery level calculator 180C may be displayed on the display unit 185C. The display unit 185C may display, in addition to displaying the battery level of the charge battery 175C, a wireless communication state (for example, transmission stable or unstable), a current mode (for example, an ultrasonic transmission/reception mode, an ultrasonic non-transmission/reception mode, or a wireless power transfer mode) of the ultrasonic diagnostic apparatus, etc. An operator (a user) may check the charge state (the battery level) of the charge battery 175C, displayed on the display unit 185C, and set the wireless power transfer mode through the input unit 225A in the ultrasonic diagnostic apparatus main body 200C. If the controller 230C in the ultrasonic diagnostic apparatus main body 200C receives a wireless power transfer setting command from the input unit 225A, the controller 230A may transfer power received from an external commercial alternating current power source 500C, to the ultrasonic probe 100C, through the wireless power transfer technique, to charge power in the charge battery 175C.

In FIG. 6B, a configuration in which the display unit 185C to display at least one of a charge state of the charge battery 175C, a wireless communication state (for example, transmission stable or unstable), and/or a current mode (for example, an ultrasonic transmission/reception mode, an ultrasonic non-transmission/reception mode, or a wireless power transfer mode) of the ultrasonic diagnostic apparatus, etc. is included in the ultrasonic probe 100C is shown as an example. However, without providing the display unit 185C in the ultrasonic probe 100C, information about a battery level (that is, a charge state) of the charge battery 175C, calculated by the battery level calculator 180C, may be transmitted to the ultrasonic diagnostic apparatus main body 200C through wireless data communication so that the display unit 200C provided in the ultrasonic diagnostic apparatus main body 200C displays a charge state of the charge battery 175C, a wireless communication state (for example, transmission stable or unstable), a current mode of the ultrasonic diagnostic apparatus, etc.

As shown in FIG. 6B, the ultrasonic diagnostic apparatus main body 200C may include a communication unit 204C. The communication unit 204C is used for wireless communication, and can transfer power to the ultrasonic probe 100C, wirelessly (wireless power transfer). The wireless power transmission is a non-contact-based system of transferring power without any contact between a power source and an electronic device, and may be implemented through any one of inductive coupling, resonant magnetic coupling, RF-based wireless power, or the like. The communication unit 204C may wirelessly transmit power received from the commercial alternating current power source 500C, to the ultrasonic probe 100C, by using a carrier frequency generated by a carrier frequency generator 203C. An antenna for transmitting or receiving electronic wave energy may be connected to the communication unit 204C.

Further, the communication unit 204C may transmit/receive various information, such as ultrasonic echo signals, a battery level of the charge battery 175C, and ultrasonic transmission/reception control signals, to/from the ultrasonic probe 100C, wirelessly, by using electronic waves (wireless data communication). However, the communication unit 204C may communicate with the ultrasonic probe 100C, using light, instead of electronic waves, wherein the light may be visible light or invisible light. The communication unit 204C may transmit ultrasonic echo signals transmitted wirelessly from the ultrasonic probe 100C, to an image processor 210C. In addition, the communication unit 204C may wirelessly transmit an ultrasonic transmission/reception control signal received from the controller 230C, to the ultrasonic probe 100C, by using a carrier frequency generated by the carrier frequency generator 203C. An antenna for transmitting or receiving electronic wave energy may be connected to the communication unit 204C.

The image processor 210C may receive ultrasonic echo signals through the communication unit 204C, and generate an ultrasonic image (or diagnosis information) of a target region in an object based on the ultrasonic echo signals. The diagnosis information may include, for example, any one or more of a B-mode image, a Color Doppler image, or a Doppler spectrum image. Various diagnosis information (ultrasonic images) for the object generated by the image processor 210C may be displayed on a display unit 215C connected to the image processor 210C. The display unit 215C may display, in addition to the various diagnosis information (for example, ultrasonic images) which relates to the object, generated by the image processor 210C, a battery level of the charge battery 175C, received from the ultrasonic probe 100C through wireless data communication, a wireless communication state (for example, transmission stable or unstable), and/or a current mode (for example, an ultrasonic transmission/reception mode, an ultrasonic non-transmission/reception mode, or a wireless power transfer mode) of the ultrasonic diagnostic apparatus, etc., received from the ultrasonic probe 100C.

The image processor 210C and the display unit 215C may be controlled by the controller 230C. Further, the controller 230C may transmit an ultrasonic transmission/reception control signal to the communication unit 204C. The input unit 225C may be electrically connected to the controller 230C. The input unit 225C may be manipulated by an operator (a user) in order to input various commands, such as a mode selection command and an ultrasonic diagnosis start command, or various information for operations of the ultrasonic diagnostic apparatus to the controller 230C.

The power supply unit 242C may convert power supplied from the external commercial alternating current power source 500C through the wired cable 201A, into a form of power that can be appropriately used to operate each of individual components (for example, the communication unit 204C, the image processor 210C, the display unit 215C, and the controller 230C) in the ultrasonic diagnostic apparatus main body 200C, and supply the converted power to the corresponding component. Further, the power supply unit 242C may transfer power supplied from the external commercial alternating current power source 500C through the wired cable 201C to the communication unit 204C so that the power can be transmitted to the ultrasonic probe 100C through wireless power transfer.

If power is no longer supplied from the external commercial alternating current power source 500C to the ultrasonic diagnostic apparatus main body 200C, for example, if a power plug is unplugged in order to move the ultrasonic diagnostic apparatus, the battery 246C may temporarily supply power to the individual components in the ultrasonic diagnostic apparatus main body 200C when the ultrasonic diagnostic apparatus enters a sleep mode or a save mode in order to store a current state as it is during movement and use the current state upon rebooting. In the sleep mode, the ultrasonic diagnostic apparatus may maintain essential functions without performing any unnecessary operation.

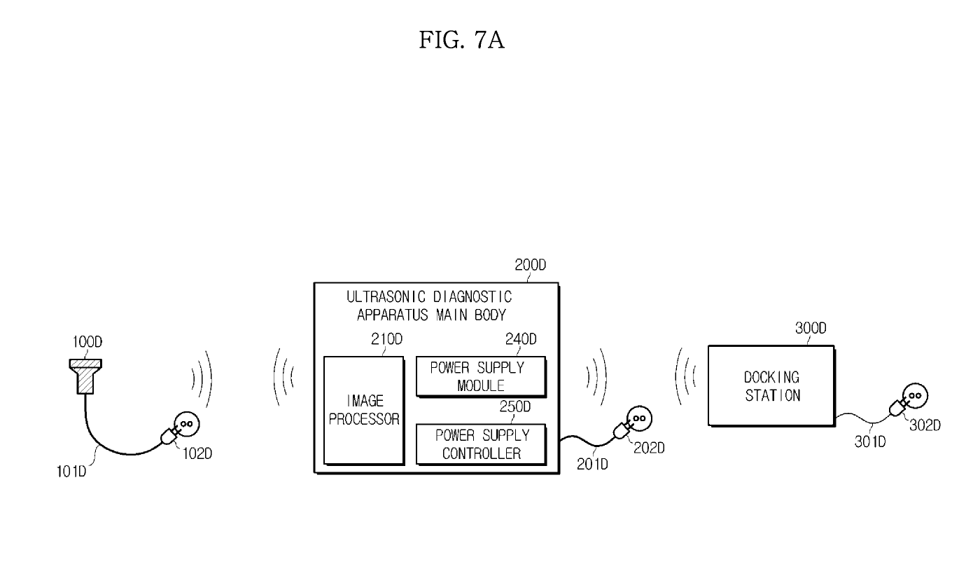

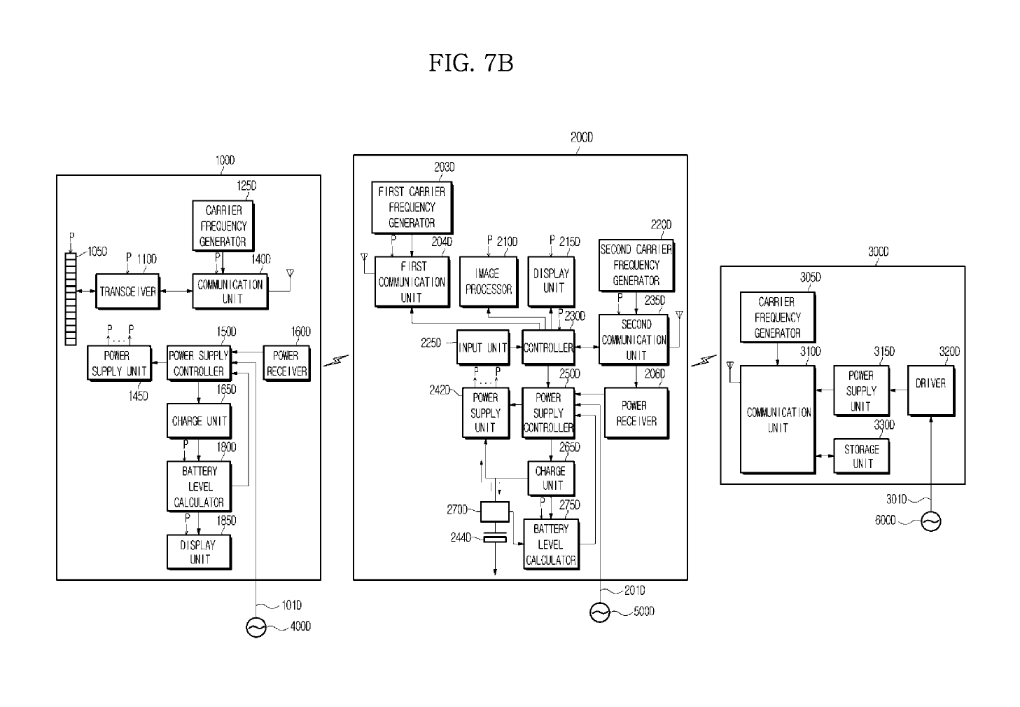

FIG. 7A is a control block diagram of an ultrasonic diagnostic system.

The above description given with reference to FIGS. 6A and 6B relates to a control configuration of an ultrasonic diagnostic apparatus according to an exemplary embodiment. In FIGS. 6A and 6B, an example in which the ultrasonic probe 100C receives power from the ultrasonic diagnostic apparatus main body 200C, wirelessly, and the ultrasonic diagnostic apparatus main body 200C receives power from the external commercial alternating current power source 500C through the wired cable 201C is shown, however, in FIG. 7A, an ultrasonic diagnostic system in which an ultrasonic probe wirelessly receives power from an ultrasonic diagnostic apparatus main body, and the ultrasonic diagnostic apparatus main body wirelessly receives power from a docking station, is shown.

As shown in FIG. 7A, the ultrasonic diagnostic system may include an ultrasonic diagnostic apparatus including an ultrasonic probe 100D and an ultrasonic diagnostic apparatus main body 200D, and a docking station 300D.

The ultrasonic probe 100D may be configured to wirelessly receive power from the ultrasonic diagnostic apparatus main body 200D. Further, the ultrasonic probe 100D may transmit ultrasonic information which relates to an object, acquired from an ultrasonic transducer array (see 105D of FIG. 7B), to the ultrasonic diagnostic apparatus main body 200D, through wireless communication. Meanwhile, a detachable power cable 101D may be connected to the ultrasonic probe 100D. One end of the detachable wired cable 101D may be connected to a power plug 102D. The ultrasonic probe 100D may receive power from an external commercial alternating current power source (see 400D of FIG. 7B) through the power plug 102D plugged in an electrical outlet. In particular, the ultrasonic probe 100D may wirelessly receive power from the ultrasonic diagnostic apparatus main body 200D, or receive power through the detachable wired power cable 101D.

The ultrasonic diagnostic apparatus main body 200D may be configured to wirelessly receive power from the docking station 300D. Further, the ultrasonic diagnostic apparatus main body 200D may transmit ultrasonic information acquired from the ultrasonic probe 100D and ultrasonic image information generated by the ultrasonic diagnostic apparatus main body 200D, to the docking station 300D, through wireless communication. Meanwhile, a detachable wired power cable 201D may be connected to the ultrasonic diagnostic apparatus main body 200D. One end of the detachable wired power cable 201D may be connected to a power plug 202D. The ultrasonic diagnostic apparatus main body 200D may receive power from an external commercial alternating current power source (see 500D of FIG. 7B) through the power plug 202D plugged in an electrical outlet. In particular, the ultrasonic diagnostic apparatus main body 200D may wirelessly receive power from the docking station 300D, and receive power through the detachable wired power cable 201D.

Further, the ultrasonic diagnostic apparatus main body 200D may wirelessly supply power to the ultrasonic probe 100D, through the wireless power transfer technique. The ultrasonic diagnostic apparatus main body 200D may receive power from the external commercial alternating current power source 500D through the power plug 202C plugged in the electrical outlet, and supply the received power to the ultrasonic probe 100C through the wireless power transfer technique. In addition, the ultrasonic diagnostic apparatus main body 200D may wirelessly receive power from the docking station 300D, and supply the received power to the ultrasonic probe 100C through the wireless power transfer technique.

The ultrasonic diagnostic apparatus main body 200D may include an image processor 210D to generate an ultrasonic image of a target region in an object based on ultrasonic echo signals received from the ultrasonic probe 100D, a power supply module 240D to supply needed power to each of components in the ultrasonic diagnostic apparatus main body 200D, and a power supply controller 250D to control power supplied from external devices (the docking station 300D and the external commercial alternating current power source 500D). The power supply module 240D may include a power supply unit 242D and a charge battery 244D (see FIG. 7B).

The docking station 300D may wirelessly supply power to the ultrasonic diagnostic apparatus main body 200D, through the wireless power transfer technique. A wired power cable 301D may be connected to the docking station 300D, and one end of the wired power cable 301D may be connected to a power plug 302D. The docking station 300D may receive power from an external commercial alternating current power source (see 600D of FIG. 7B) through the power plug 302D plugged in the electrical outlet, and supply the received power to the ultrasonic diagnostic apparatus main body 200D through the wired power transfer technique.

FIG. 7B is a control block diagram illustrating configurations of the ultrasonic probe 100D, the ultrasonic diagnostic apparatus main body 200D, and the docking station 300D shown in FIG. 7A.

Since components of the ultrasonic probe 100D as shown in FIG. 7B are the same as those of the ultrasonic probe 100C as shown in FIG. 6B, detailed descriptions for the components of the ultrasonic probe 100D will be omitted.

Further, since components of the ultrasonic diagnostic apparatus main body 200D as shown in FIG. 7B are the same as those of the ultrasonic diagnostic apparatus main body 200A shown in FIG. 4B, except that a first communication unit 204D to transfer power from the ultrasonic diagnostic apparatus main body 200D to the ultrasonic probe 100D, wirelessly and to communicate data wirelessly between the ultrasonic diagnostic apparatus main body 200D and the ultrasonic probe 100D, and a second carrier frequency generator 203D to generate a carrier frequency used for wireless power transfer and wireless data communication are further included in the ultrasonic diagnostic apparatus main body 200D, detailed descriptions for the components in the ultrasonic diagnostic apparatus main body 200D will be omitted.

In addition, since components in the docking station 300D as shown in FIG. 7B are the same as those of the docking station 300A as shown in FIG. 4B, detailed descriptions for the docking station 300D will be omitted.

The exemplary embodiments described above with reference to FIGS. 4A to 7B can be applied to the cart type ultrasonic diagnostic apparatus as shown in FIG. 1 or to the portable ultrasonic diagnostic apparatus as shown in FIG. 2.

FIG. 8A is a control block diagram of an ultrasonic diagnostic system.

In the exemplary embodiments as described above, an ultrasonic diagnostic system (see FIGS. 4A, 4B, 5A, 5B, 7A, and 7B) implemented such that an ultrasonic diagnostic apparatus including an ultrasonic probe and an ultrasonic diagnostic apparatus main body can wirelessly receive power from a docking station, and an ultrasonic diagnostic apparatus (see FIGS. 6A and 6B) implemented such that a ultrasonic probe can wirelessly receive power from an ultrasonic diagnostic apparatus main body, have been described. In the following description, an ultrasonic diagnostic system implemented such that an ultrasonic probe can receive power from a docking station wirelessly will be described in detail with reference to FIGS. 8A and 8B.

As shown in FIG. 8A, the ultrasonic diagnostic system may include an ultrasonic probe 100E and a docking station 300E.

The ultrasonic probe 100E as shown in FIG. 8A may include an ultrasonic transducer array (see 105E of FIG. 8B) configured to transmit and receive ultrasonic signals, an image processor (see 115E of FIG. 8B) configured to generate an ultrasonic image based on the received ultrasonic echo signals, a display unit 120E configured to display the generated ultrasonic image, and a controller (see 135E of FIG. 8B) configured to control overall operations of the ultrasonic probe 100E so that the ultrasonic probe 100E itself constitutes an ultrasonic diagnostic apparatus. In particular, since the ultrasonic probe 100E includes all essential components (that is, components related to ultrasonic transmission/reception and image processing) needed to perform an ultrasonic diagnosis, the ultrasonic probe 100E can be used to diagnose a target region in an object.

The ultrasonic probe 100E may wirelessly receive power from the docking station 300E. Further, the ultrasonic probe 100E may transmit ultrasonic information for an object acquired by the ultrasonic transducer array and various diagnosis information (ultrasonic images) for the object generated by the image processor, to the docking station 300E, through wireless communication. Meanwhile, a detachable wired power cable 101E may be connected to the ultrasonic probe 100E. One end of the detachable wired power cable 101E may be connected to a power plug 102E. The ultrasonic probe 100E may receive power from an external commercial alternating current power source (see 400E of FIG. 8B) through the power plug 102E plugged in an electrical outlet. In particular, the ultrasonic probe 100E may wirelessly receive power from the docking station 300E, and receive power through the detachable wired power cable 101E.

The docking station 300E may supply power to the ultrasonic probe wirelessly through the wireless power transfer technique. A wired power cable 301E may be connected to the docking station 300E, and one end of the wired power cable 301E may be connected to a power plug 302E. The docking station 300E may receive power from an external commercial alternating current power source (see 600E of FIG. 8B) through the power plug 302E plugged in an electrical outlet, and supply the received power to the ultrasonic probe 100E through the wireless power transfer technique.

FIG. 8B is a control block diagram illustrating configurations of the ultrasonic probe 100E and the docking station 300E shown in FIG. 8A.

As shown in FIG. 8B, the ultrasonic probe 100E may include an ultrasonic transducer array 105E in which a plurality of ultrasonic transducers are arranged in an array.

The ultrasonic transducer array 105E may be electrically connected to a transceiver 110E. The transceiver 110E may transmit a driving signal to the ultrasonic transducer array 105E so that the ultrasonic transducer array 105E irradiates ultrasonic waves to a target region in an object. Further, the transceiver 110E may receive ultrasonic echo signals reflected from the target region in the object from the ultrasonic transducer array 105E. The transceiver 110E may be electrically connected to a controller 135E. The transceiver 110E may transmit or receive ultrasonic waves based on an ultrasonic transmission/reception control signal received from the controller 135E. Further, the transceiver 110E may transfer ultrasonic echo signals received from the ultrasonic transducer array 105E to the image processor 115E.

The image processor 115E may receive ultrasonic echo signals from the transceiver 110E, and generate an ultrasonic image (or diagnosis information) of a target region in an object based on the ultrasonic echo signals. The diagnosis information (for example, an ultrasonic image) for the object, generated by the image processor 115E, may be displayed on a display unit 120E connected to the image processor 115E.

The image processor 115E and the display unit 120E may be controlled by the controller 135E. Further, the controller 135E may transfer an ultrasonic transmission/reception control signal to the transceiver 110E. An input unit 130E may be electrically connected to the controller 135E. The input unit 130E may be manipulated by an operator (a user) in order for the operator to input various commands, such as a mode selection command and an ultrasonic diagnosis start command, or various information related to operations of the ultrasonic diagnostic apparatus, to the controller 135E.

The controller 135E may be electrically connected to a communication unit 140E. The controller 135E may transmit ultrasonic echo signals, received from the transceiver 110E, and various information, such as an ultrasonic image (diagnosis information) for an object, received from the image processor 115E, to the docking station 300E, through the communication unit 140E.

The communication unit 140E is used for wireless communication. For example, the communication unit 140A may wirelessly transmit/receive various information, such as ultrasonic echo signals and ultrasonic images (diagnosis information), to/from the docking station 300E, by using electronic waves (wireless data communication). However, the communication unit 140E may communicate with the docking station 300E, using light, instead of electronic waves, wherein the light may be visible light or invisible light. The communication unit 140E may wirelessly transmit various information, such as the ultrasonic echo signals and the ultrasonic images (diagnosis information), to the docking station 300E, by using a carrier frequency generated by a carrier frequency generator 125E. An antenna for transmitting or receiving electronic wave energy may be connected to the communication unit 140E.

Further, the communication unit 140E may wirelessly receive power from the docking station 300E (wireless power transfer). The wireless power transfer is a non-contact-based system of transferring power without any physical contact between a power source and an electronic device, and may be implemented through any of inductive coupling, resonant magnetic coupling, RF-based wireless power, or the like. The communication unit 140E may transfer power received from the docking station 300E to a power receiver 160E.

At this time, an arbitrary frequency in an ultrasonic frequency band may be set to a carrier frequency for wireless power transfer. In this case, in an ultrasonic non-transmission/reception mode (for example, a freeze mode), wireless data communication or wireless power transfer may be performed using ultrasonic pulses generated from the ultrasonic transducer array 105E. In the case in which an arbitrary frequency in an ultrasonic frequency band is set to a carrier frequency for wireless data communication or wireless power transfer, the carrier frequency generator 125E may be omitted.

The power receiver 160E may receive power supplied through the wireless power transfer technique. The power receiver 160E may receive power supplied wirelessly through the inductive method or the like, and transfer the received power to the power supply controller 150E.

The power supply controller 150E may control power supplied from external devices (the docking station 300E and the external commercial alternating current power source 400E). For example, the power supply controller 150E may be a switch. If the power supply controller 150E receives power from the external commercial alternating current power source 400E through the detachable wired power cable 101E, the power supply controller 150E may transfer the received power to the power supply unit 145E. The power supply unit 145E may convert the power received through the power supply controller 150E into a form of power that can be appropriately used to operate each of individual components (for example, the ultrasonic transducer array 105E, the transceiver 110E, the image processor 115E, the display unit 120E, the controller 135E, etc.) in the ultrasonic probe 100E, and supply the converted power to the corresponding component.

Meanwhile, if the power supply controller 150E receives power from the power receiver 160E, the power supply controller 150E may transfer the received power to a charge unit 165E. A charge battery 175E may be charged by the charge unit 165E. The charge unit 165E may charge power received through the power receiver 160E and the power supply controller 150E in the charge battery 175E. If the power supply controller 150E receives a wireless power transfer mode setting command from an operator (a user) through an input unit 225E of the ultrasonic diagnostic apparatus main body 200E, the power supply controller 150E may enter a wireless power transfer mode to charge power supplied from the power receiver 160E in the charge battery 175E, or in an ultrasonic non-transmission/reception mode (for example, a freeze mode), the power supply controller 150E may be automatically switched to the wireless power transfer mode (automatic mode switching) to charge power supplied from the power receiver 160E in the charge battery 175E. The charge battery 175E may be charged through any of a capacitive method using an electric field, a resonance method using a magnetic field, or a inductive method. The power supply unit 145E may convert power that is accumulated in the charge battery 175E into a form of power that can be appropriately used to operate each of the individual components (for example, the ultrasonic transducer array 105E, the transceiver 110E, the image processor 115E, the display unit 120E, the controller 135E, etc.) in the ultrasonic probe 100E, and supply the converted power to the corresponding component.

The charge battery 175E may be a primary battery or a secondary battery. If the charge battery 175E is a secondary battery, it is possible to separate the charge battery 175E from the ultrasonic probe 100E and then charge power in the charge battery 175E.

A current sensor 170E may be connected in series to the charge battery 175E. The current sensor 170E may detect an amount and direction of current. Information detected by the current sensor 175E may be transferred to a battery level calculator 180E. The battery level calculator 180E may accumulatively add current entering the charge battery 175E over time to calculate a charge amount, accumulatively add current discharged from the charge battery 175E over time to calculate a discharge amount, and then calculate a battery level of the charge battery 175E based on a difference between the charge amount and the discharge amount. The battery level of the charge battery 175E, calculated by the battery level calculator 180E may be displayed on a display unit 185E. The display unit 185E may display, in addition to displaying the battery level of the charge battery 175E, a wireless communication state (for example, transmission stable or unstable), a current mode (for example, an ultrasonic transmission/reception mode, an ultrasonic non-transmission/reception mode, or a wireless power transfer mode) of the ultrasonic diagnostic system, etc. An operator (a user) may check the charge state (the battery level) of the charge battery 175E, displayed on the display unit 185E, and set the wireless power transfer mode through the input unit 225E. If the controller 135E receives a wireless power transfer setting command from the input unit 120E, the controller 135E may control the communication unit 140E and the power supply controller 150E to receive power from the docking station 300E through wireless power transfer and charge the power in the charge battery 175E.

As shown in FIG. 8B, the docking station 300E may include a power supply unit 315E. The power supply unit 315E may supply power to the power receiver 160E in the ultrasonic probe 100E through the inductive method or the like. The power supply unit 315E may be driven by a driver 320E. The driver 320E may be connected to the external commercial alternating current power source 600E through a wired power cable 301E. The driver 320E may transfer power received from the external commercial alternating current power source 600E to the power supply unit 315E.

Meanwhile, the power supply unit 315E may be electrically connected to a communication unit 310E. The power supply unit 315E may transfer power received from the driver 320E to the communication unit 310E.

The communication unit 310E is used for wireless communication. For example, the communication unit 310E may wirelessly transmit power to the ultrasonic probe 100E (wireless power transfer). The wireless power transfer is a non-contact-based system of transferring power without any physical contact between a power source and an electronic device, and may be implemented through any of inductive coupling, resonant magnetic coupling, RF-based wireless power, or the like. The communication unit 310E may wirelessly transfer power received from the external commercial alternating current power source 600E, to the ultrasonic probe 100E, by using a carrier frequency generated by a carrier frequency generator 305E. An antenna for transmitting or receiving electronic wave energy may be connected to the communication unit 310E.

Further, the communication unit 310E may wirelessly transmit/receive ultrasonic echo signals or ultrasonic images (diagnosis information) to/from the ultrasonic probe 100E, by using electronic waves (wireless data communication). However, the communication unit 310E may communicate with the ultrasonic probe 100E using light, instead of electronic waves, wherein the light may be visible light or invisible light. Various information, such as ultrasonic echo signals and ultrasonic images (diagnosis information), transmitted wirelessly from the ultrasonic probe 100E through the communication unit 310E may be stored in a storage unit 330E.

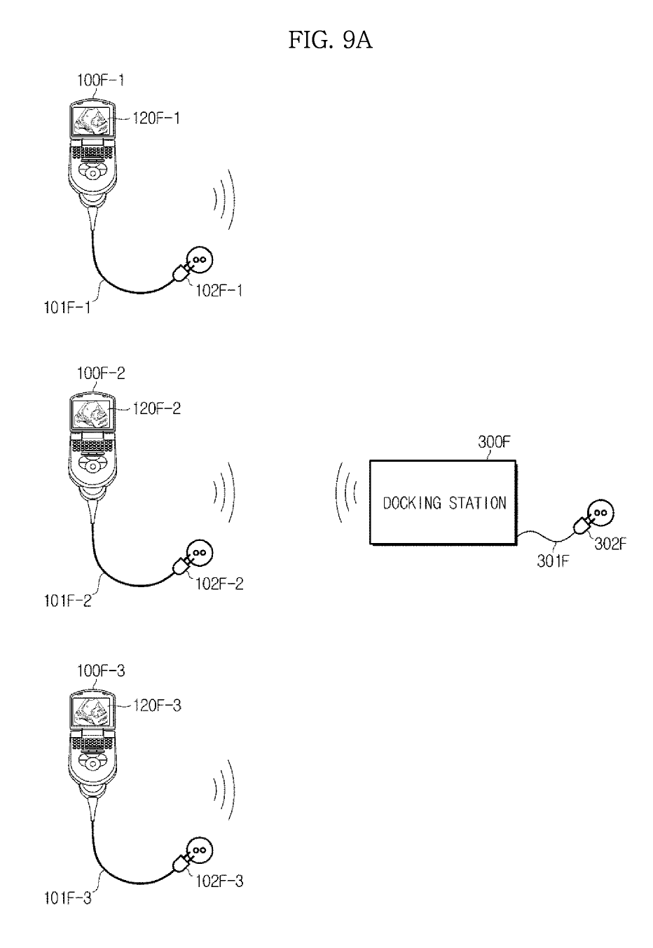

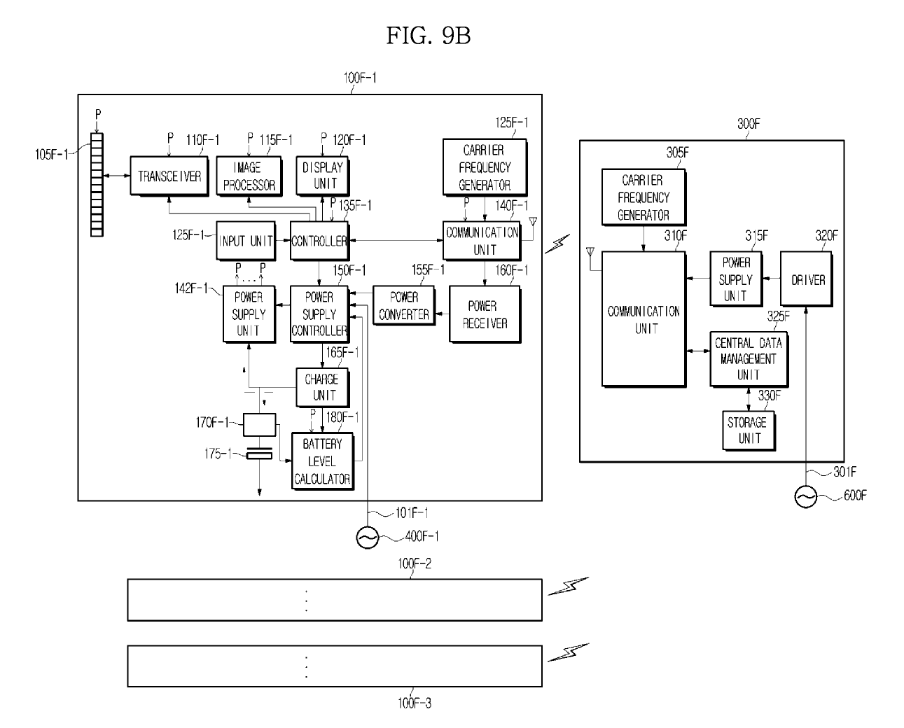

FIG. 9A is a control block diagram of an ultrasonic diagnostic system.

The above description given with reference to FIGS. 8A and 8B relate to a control configuration of an ultrasonic diagnostic system according to an exemplary embodiment. In FIGS. 8A and 8B, a system in which the ultrasonic probe 100E, which itself is capable of functioning as an ultrasonic diagnostic apparatus, wirelessly receives power from the docking station 300E, is shown, however, in FIG. 9A, an ultrasonic diagnostic system in which a plurality of ultrasonic probes, each of which is capable of functioning as an ultrasonic diagnostic apparatus, wirelessly receive power from a docking station, is shown.

As shown in FIG. 9A, the ultrasonic diagnostic system may include a plurality of ultrasonic probes 100E-1, 100E-2, and 100E-3, and a docking station 300F.

Each of the ultrasonic probes 100E-1, 100E-2, and 100E-3 may wirelessly receive power from the docking station 300. Further, each of the ultrasonic probes 100E-1, 100E-2, and 100E-3 may transmit ultrasonic information for an object, acquired by each of a plurality of ultrasonic transducer arrays (see 105F of FIG. 9B), and various diagnosis information (ultrasonic images) which relates to the object, generated by an image processor (see 115F of FIG. 9B), to the docking station 300F, through wireless communication. Meanwhile, a plurality of detachable wired cables 101F-1, 101F-2, and 101F-3 may be connected to the respective ultrasonic probes 100E-1, 100E-2, and 100E-3. One ends of the detachable wired cables 101F-1, 101F-2, and 101F-3A may be connected to a plurality of power plugs 102F-1, 102F-2, and 102F-3. The respective ultrasonic probes 100E-1, 100E-2, and 100E-3 may receive power from an external commercial alternating current power source (see 400E-1 of FIG. 9B) through the respective power plugs 102F-1, 102F-2, and 102F-3 plugged in electrical outlets. In particular, the respective ultrasonic probes 100E-1, 100E-2, and 100E-3 may receive power from the docking station 300F, wirelessly, or receive power through the respective detachable wired power cables 101F-1, 101F-2, and 101F-3.

The docking station 300F may wirelessly supply power to the respective ultrasonic probes 100E-1, 100E-2, and 100E-3, through the wireless power transfer technique. A wired power cable 301F may be connected to the docking station 300F, and one end of the wired power cable 301F may be connected to a power plug 302F. The docking station 300F may receive power from an external commercial alternating current power source (see 600F of FIG. 9B) through the power plug 302F plugged in an electrical outlet, and supply the received power to the respective ultrasonic probes 100E-1, 100E-2, and 100E-3 through the wireless power transfer technique.

FIG. 9B is a control block diagram illustrating configurations of the ultrasonic probes 100E-1, 100E-2, and 100E-3 and the docking system 300F shown in FIG. 9A.

Since the ultrasonic probes 100E-1, 100E-2, and 100E-3 have the same configuration, in FIG. 9B, a configuration of the first ultrasonic probe 100E-1 is shown in detail, and configurations of the second and third ultrasonic probes 100E-2 and 100E-3 are not shown.

Further, the configuration of each of the ultrasonic probes 100E-1, 100E-2, and 100E-3 as shown in FIG. 9B is the same as the configuration of the ultrasonic probe 100E as shown in FIG. 8B, except that the ultrasonic probes 100E-1, 100E-2, and 100E-3 further include a plurality of power converters 155F-1, 155F-2, and 155F-3 configured to convert power supplied from the docking station 300B into a form of power that can be appropriately used by the respective ultrasonic probes 100E-1, 100E-2, and 100E-3. Accordingly, in the following description, detailed descriptions for the individual components in the ultrasonic probes 100E-1, 100E-2, and 100E-3 will be omitted.

As shown in FIG. 9B, the docking station 300F may include a power supply unit 315F. The power supply unit 315F may supply power to a power receiver 160E-1 in each of the ultrasonic probes 100E-1, 100E-2, and 100E-3 through the inductive method or the like. The power supply unit 315F may be driven by a driver 320F. The driver 320F may be connected to an external commercial alternating current power source 600F through the wired power cable 301F. The driver 320F may transfer power received from the external commercial alternating power source 600F to the power supply unit 315F.

Meanwhile, the power supply unit 315F may be electrically connected to the communication unit 310F. The power supply unit 315F may transfer power received from the driver 320F to the communication unit 310F.

The communication unit 310F is used for wireless communication. For example, the communication unit 310F may wirelessly transmit power to the ultrasonic probes 100E-1, 100E-2, and 100E-3 (wireless power transfer). The wireless power transfer is a non-contact-based system of transferring power without any physical contact between a power source and an electronic device, and may be implemented through any of inductive coupling, resonant magnetic coupling, RF-based wireless power, or the like. The communication unit 310F may wirelessly transmit power supplied from the external commercial alternating current power source 600F, to the ultrasonic probes 100E-1, 100E-2, and 100E-3, by using a carrier frequency generated by the carrier frequency generator 305F. An antenna for transmitting or receiving electric wave energy may be connected to the communication unit 310F.

Further, the communication unit 310F may wirelessly transmit/receive various information, such as ultrasonic echo signals and ultrasonic images (diagnosis information), to/from the ultrasonic probes 100E-1, 100E-2, and 100E-3, by using electric waves (wireless data communication). However, the communication unit 310F may communicate with the ultrasonic probes 100E-1, 100E-2, and 100E-3 using light, instead of electronic waves, wherein the light may be visible light or invisible light. Various information, such as ultrasonic echo signals and ultrasonic images (diagnosis information), transmitted wirelessly from the ultrasonic probes 100E-1, 100E-2, and 100E-3 through the communication unit 310F may be transferred to a central data management unit 325F.

The central data management unit 325F may manage various information received wirelessly from the ultrasonic probes 100E-1, 100E-2, and 100E-3. The central data management unit 325F may store information needed to be stored, among various information received wirelessly from the ultrasonic probes 100E-1, 100E-2, and 100E-3, in a storage unit 330F. Further, the central data management unit 325F may read, when receiving a data transfer request from each ultrasonic probe 100E-1, 100E-2, or 100E-3, the various information stored in the storage unit 330F, and wirelessly transmit the read information to the ultrasonic probe 100E-1, 100E-2, or 100E-3, through the communication unit 310F.

As shown in FIGS. 9A and 9B, in the ultrasonic diagnostic system in which the plurality of ultrasonic probes 100E-1, 100E-2, and 100E-3 wirelessly receive power from the docking station 300F, the docking station 300F functions as a hub for power supply. In the ultrasonic diagnostic system in which data is transmitted/received wirelessly between the plurality of ultrasonic probes 100E-1, 100E-2, and 100E-3 and the docking station 300F, the docking station 300F may also function as a data hub.

The exemplary embodiments described above with reference to FIGS. 8A to 9B can be applied to the handheld ultrasonic diagnostic apparatus (an ultrasonic probe or an ultrasonic probe handle) as shown in FIGS. 3A and 3B.

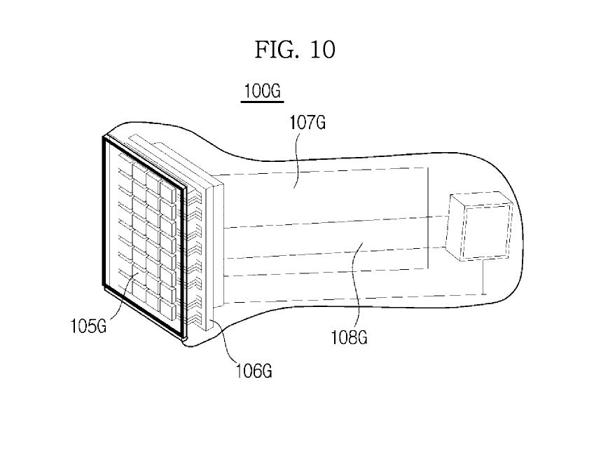

FIG. 10 illustrates an internal structure of an ultrasonic probe. In FIG. 10, an ultrasonic probe including an electronic circuit, such as a transceiver or an image processor, as shown in FIGS. 6B, 7B, 8B, and 9B, is shown.

Generally, an electronic circuit includes a plurality of active elements, and such active elements are amplified or oscillated by receiving energy from an external device so that a heating phenomenon occurs. Accordingly, an ultrasonic probe including an electronic circuit requires a heat-emitting and cooling module to emit generated heat to the outside.

As shown in FIG. 10, an ultrasonic probe 100G may include an ultrasonic transducer array 105G, an electronic circuit unit 106G, a heat sinking plate 107G, and a cooling fin 108G.

The ultrasonic transducer array 105G is configured by arranging a plurality of ultrasonic transducers in an array. The ultrasonic transducer may include any one or more of a magnetostrictive ultrasonic transducer using the magnetostrictive effect of a magnetic material, a piezoelectric ultrasonic transducer using the piezoelectric effect of a piezoelectric material, a capacitive micromachined ultrasonic transducer (CMUT) that transmits and receives ultrasonic waves using vibration of several hundreds or thousands of micromachined thin films, a Piezoelectric Micromachined Ultrasonic Transducer (pMUT), and/or a single crystal.

The electronic circuit unit 106G is a circuit which is configured to generate an ultrasonic image of an object based on received/transmitted ultrasonic waves or ultrasonic echo signals. The electronic circuit unit 106G causes the heating phenomenon.

The heat sinking plate 107G may emit heat generated in the ultrasonic probe 100G due to the electronic circuit unit 106G to the outside. The heat sinking plate 107G may be made of a metal material, such as, for example, aluminum. The cooling fin 108G may cool the ultrasonic probe 100G using air inflowing from the outside. The cooling fin 108G may have a pleated shape formed by maximally widening a surface area in order to improve a cooling effect. The cooling fin 108G may also be made of a metal material such as aluminum.

The ultrasonic probe as shown in FIGS. 6B, 7B, 8B, and 9B may include an antenna connected to a communication unit which is configured for wireless data communication or wireless power transfer, however, as shown in FIG. 10, if the ultrasonic probe 100G includes the heat sinking plate 107G or the cooling fin 1008 made of a metal material, the heat sinking plate 107G or the cooling fin 108G may function as an antenna for wireless data communication or wireless power transfer.

Therefore, according to the ultrasonic probe and the ultrasonic diagnostic apparatus as described above, it is possible to efficiently supply power to the ultrasonic probe and the ultrasonic diagnostic apparatus main body regardless of time and place, by applying the wireless power transfer technique to the ultrasonic probe and the ultrasonic diagnostic apparatus main body.

Further, according to the ultrasonic probe and the ultrasonic diagnostic apparatus as described above, it is possible to improve mobility and portability of the ultrasonic probe and the ultrasonic diagnostic apparatus main body and increasing use times of the ultrasonic probe and the ultrasonic diagnostic apparatus main body, by applying the wireless power transfer technique to the ultrasonic probe and the ultrasonic diagnostic apparatus main body.

In addition, according to the ultrasonic probe and the ultrasonic diagnostic apparatus as described above, it is possible to install charge batteries of smaller volumes in the ultrasonic probe and the ultrasonic diagnostic apparatus main body to reduce sizes and weights of the ultrasonic probe and the ultrasonic diagnostic apparatus main body, by applying the wireless power transfer technique to the ultrasonic probe and the ultrasonic diagnostic apparatus main body.

Although a few exemplary embodiments have been shown and described, it will be appreciated by those of skill in the art that changes may be made in these exemplary embodiments without departing from the principles and spirit of the present disclosure, the scope of which is defined in the claims and their equivalents.

What is claimed is:

1. An ultrasonic diagnostic apparatus comprising:
an ultrasonic probe which includes an ultrasonic transducer array; and
an ultrasonic diagnostic apparatus main body comprising a transceiver configured to transmit and receive ultrasonic waves via the ultrasonic transducer array, an image processor configured to generate an ultrasonic image of an object based on an ultrasonic echo signal acquired via the transceiver, a communicator configured to wirelessly communicate with a docking station, and a charger configured to charge power which is wirelessly received from the docking station via the communicator, in a charge battery.

2. The ultrasonic diagnostic apparatus according to claim 1, wherein the ultrasonic diagnostic apparatus main body further comprises a power supply controller configured to control power supplied from an external device, and
wherein the power supply controller is further configured to receive power which is transmitted wirelessly from the docking station, and to transfer the received power to the charger.

3. The ultrasonic diagnostic apparatus according to claim 1, wherein the communicator is further configured to wirelessly transmit the ultrasonic echo signal and the ultrasonic image to the docking station.

4. The ultrasonic diagnostic apparatus according to claim 1, wherein the charger is further configured to charge the power received from the docking station, in the charge battery, by using at least one method from among a capacitive method using an electric field, a resonance method using a magnetic field, and an inductive method.

5. The ultrasonic diagnostic apparatus according to claim 1, wherein the ultrasonic diagnostic apparatus main body further comprises:
a battery level calculator configured to calculate a battery level of the charge battery; and
a display configured to display the calculated battery level of the charge battery and the ultrasonic image.

6. The ultrasonic diagnostic apparatus according to claim 1, wherein the ultrasonic diagnostic apparatus main body further comprises an input device configured to set a wireless power transfer mode for wirelessly receiving power from the docking station.

7. An ultrasonic diagnostic apparatus, comprising an ultrasonic probe and an ultrasonic diagnostic apparatus main body,
wherein the ultrasonic probe comprises an ultrasonic transducer array, a transceiver configured to transmit and receive ultrasonic waves via the ultrasonic transducer array, a first communicator configured to wirelessly communicate with the ultrasonic diagnostic apparatus main body, and a charger configured to charge power which is wirelessly received from the ultrasonic diagnostic apparatus main body via the first communicator, in a charge battery, and
wherein the ultrasonic diagnostic apparatus main body comprises a second communicator configured to wirelessly communicate with the ultrasonic probe, and an image processor configured to generate an ultrasonic image of an object based on an ultrasonic echo signal acquired via wireless communication with the ultrasonic probe.

8. The ultrasonic diagnostic apparatus according to claim 7, wherein the ultrasonic probe further comprises a power supply controller configured to control power supplied from an external device, and
wherein the power supply controller is further configured to receive power which is transmitted wirelessly from the ultrasonic diagnostic apparatus main body, and to transfer the received power to the charger.

9. The ultrasonic diagnostic apparatus according to claim 7, wherein the first communicator is further configured to wirelessly transmit the ultrasonic echo signal to the ultrasonic diagnostic apparatus main body.

10. The ultrasonic diagnostic apparatus according to claim 7, wherein the ultrasonic probe further comprises:
a battery level calculator configured to calculate a battery level of the charge battery; and
a display configured to display the calculated battery level of the charge battery.

11. An ultrasonic diagnostic apparatus, comprising an ultrasonic probe and an ultrasonic diagnostic apparatus main body,
wherein the ultrasonic probe comprises an ultrasonic transducer array, a transceiver configured to transmit and receive ultrasonic waves via the ultrasonic transducer array, a probe communicator configured to wirelessly communicate with the ultrasonic diagnostic apparatus main body, and a probe charger configured to charge power which is wirelessly received from the ultrasonic diagnostic apparatus main body via the probe communicator, in a probe charge battery, and
wherein the ultrasonic diagnostic apparatus main body comprises a first main body communicator configured to wirelessly communicate with the ultrasonic probe, an image processor configured to generate an ultrasonic image of an object based on an ultrasonic echo signal acquired from the ultrasonic probe via the first main body communicator, a second main body communicator configured to wirelessly communicate with a docking station, and a main body charger configured to charge power which is wirelessly received from the docking station via the second main body communicator, in a main body charge battery.

12. The ultrasonic diagnostic apparatus according to claim 11, wherein the ultrasonic probe further comprises a probe power supply controller configured to control power supplied from an external device, and
wherein the probe power supply controller is further configured to receive power which is transmitted wirelessly from the ultrasonic diagnostic apparatus main body, and to transfer the received power to the probe charger.

13. The ultrasonic diagnostic apparatus according to claim 11, wherein the ultrasonic diagnostic apparatus main body further comprises a main body power supply controller configured to control power supplied from an external device, and
wherein the main body power supply controller is further configured to receive power which is transmitted wirelessly from the docking station, and to transfer the received power to the main body charger.

14. The ultrasonic diagnostic apparatus according to claim 11, wherein the probe communicator is further configured to wirelessly transmit the ultrasonic echo signal to the ultrasonic diagnostic apparatus main body.

15. The ultrasonic diagnostic apparatus according to claim 14, wherein the second main body communicator is further configured to wirelessly transmit the ultrasonic echo signal and the ultrasonic image to the docking station.

16. The ultrasonic diagnostic apparatus according to claim 11, wherein the ultrasonic probe further comprises:
a probe battery level calculator configured to calculate a battery level of the probe charge battery; and
a probe display configured to display the calculated battery level of the probe charge battery.

17. The ultrasonic diagnostic apparatus according to claim 11, wherein the ultrasonic diagnostic apparatus main body further comprises:
a main body battery level calculator configured to calculate a battery level of the main body charge battery; and
a main body display configured to display the calculated battery level of the main body charge battery.

18. An ultrasonic probe comprising:
an ultrasonic transducer array;
a transceiver configured to transmit and receive ultrasonic waves via the ultrasonic transducer array;
an image processor configured to generate an ultrasonic image of an object based on an ultrasonic echo signal acquired via the transceiver;
a display configured to display the ultrasonic image of the object; and
a communicator configured to wirelessly communicate with a docking station; and
a charger configured to charge power which is wirelessly received from the docking station via the communicator, in a charge battery.

19. The ultrasonic probe according to claim 18, further comprising a power supply controller configured to control power supplied from an external device,
wherein the power supply controller is further configured to receive power which is transmitted wirelessly from the docking station, and to transfer the received power to the charger.

20. The ultrasonic probe according to claim 18, wherein the communicator is further configured to wirelessly transmit the ultrasonic echo signal and the ultrasonic image to the docking station.
